(19) United States
(12) Patent Application Publication
(10) Pub. No.: US 2008/0038192 A1
(43) Pub. Date: Feb. 14, 2008

(54) Diagnostic Methods Of Multiple Organ Amyloidosis

(75) Inventor: Francine Gervais, Ile Bizard (CA)

Correspondence Address: LAHIVE & COCKFIELD, LLP, ONE POST OFFICE SQUARE, BOSTON, MA, 02109-2127, US

(73) Assignee: NEUROCHEM INC., LAVAL (CA)

(21) Appl. No.: 11/632,179

(22) PCT Filed: Jul. 18, 2005
(86) PCT No.: PCT/IB05/03418

§ 371 Date: Jan. 16, 2007

Related U.S. Application Data
(60) Provisional application No. 60/589,411, filed on Jul. 19, 2004.

(51) Int. Cl. G01N 33/53; C12Q 1/68; G01N 33/543

(52) U.S. Cl. 424/1.49; 435/6; 435/7.21; 435/7.92; 436/501; 436/518

(57) ABSTRACT

Described are methods of assessing whether a subject has or is at risk of having multiple organ amyloidosis (MOA) The method includes detecting a diagnostically predictive collection of biomarkers of multiple organ amyloidosis, wherein the detection of a diagnostically predictive collection of biomarkers indicates the subject has or is at risk of having multiple organ amyloidosis Also described are methods of monitoring treatment of subjects with multiple organ amyloidosis and evaluating therapeutic compounds Representative biomarkers for use in the methods may be selected from variant serum amyloid A (SAA) allele, elevated SAA level, elevated C-reactive protein (CRP) level, depressed glycosammoglycan (GAG) level, elevated interleukin-18 (IL-18) level, elevated macrophage-colony stimulating factor (M-CSF) level, elevated hepatocyte growth factor (HGF) level, presence of an antibody against citrullmated vimentm (Sa), presence of a monoclonal immunoglobulin light chain, increased serum albumin, and increased creatinine clearance

### RELATED APPLICATIONS

This application claims the benefit of U.S. Patent Application Ser. No. 60/589,411 filed Jul. 19, 2004, the contents of which is hereby incorporated by reference.

### BACKGROUND

Amyloidosis refers to a pathological condition characterized by the presence of amyloid fibrils. Amyloid is a generic term referring to a group of diverse but specific protein deposits (intracellular or extracellular) which are seen in a number of different diseases. Though diverse in their occurrence, all amyloid deposits have common morphologic properties, stain with specific dyes (e.g., Congo red), and have a characteristic red-green birefringence appearance in polarized light after staining. They also share common ultrastructural features and common X-ray diffraction and infrared spectra.

Amyloid-related diseases can either be restricted to one organ or spread to several organs. The first instance is referred to as localized amyloidosis while the second is referred to as systemic amyloidosis.

Some amyloidotic diseases can be idiopathic, but most of these diseases appear as a complication of a previously existing disorder. For example, primary amyloidosis, e.g., AL amyloidosis, can appear without any other pathology or can follow plasma cell dyscrasia or multiple myeloma.

Localized amyloidoses are those that tend to involve a single organ system. Different amyloids are also characterized by the type of protein present in the deposit. For example, neurodegenerative diseases such as scrapie, bovine spongiform encephalitis, Creutzfeldt-Jakob disease, and the like are characterized by the appearance and accumulation of a protease-resistant form of a prion protein (referred to as AScr or PrP-27) in the central nervous system. Similarly, Alzheimer's disease, another neurodegenerative disorder, is characterized by neuritic plaques and neurofibrillary tangles. In this case, the plaque and blood vessel amyloid is formed by the deposition of fibrillar Aβ amyloid protein. Other diseases such as adult-onset diabetes (type II diabetes) are characterized by the localized accumulation of amyloid in the pancreas. In dialysis-related amyloidosis, plaques composed of β2 microglobulin develop in the carpal tunnel and in collagen-rich tissues of the joints.

Secondary amyloidosis, also referred to as AA amyloidosis, is usually seen associated with chronic infection (such as tuberculosis) or chronic inflammation (such as rheumatoid arthritis). A familial form of secondary amyloidosis is also seen in Familial Mediterranean Fever (FMF). This familial type of amyloidosis, as are the other types of familial amyloidosis, is genetically inherited and is found in specific population groups. In both primary and secondary amyloidosis, deposits are found in several organs and are thus considered systemic amyloid diseases.

Another type of systemic amyloidosis is found in long-term hemodialysis subjects. In each of these cases, a different amyloidogenic protein is involved in amyloid deposition.

### SUMMARY OF THE INVENTION

Amyloidosis disorders, such as for example, amyloidoses affecting multiple organs, e.g., AA amyloidosis and AL amyloidosis, referred to herein as multiple organ amyloidosis or MOA, are progressive and fatal conditions. AA amyloidosis often occurs in a proportion of subjects with chronic inflammatory diseases, such as rheumatoid arthritis (RA), ankylosing spondylitis, juvenile rheumatoid arthritis, and Crohn's disease. Subjects with RA represent about 50% of the thousands of subjects suffering from AA Amyloidosis. The disease also occurs in subjects suffering from many other conditions ranging from chronic infections to inherited inflammatory diseases such as Familial Mediterranean Fever (FMF). AL amyloidosis often occurs in subjects with multiple myeloma or a plasma cell dyscrasia.

Multiple organ amyloidosis is insidious and progressive. Symptoms tend to present in the later stages of the disease and as a result, the disease frequently remains undiagnosed until significant organ damage has occurred. Amyloid fibrils, e.g., AA or AL fibrils, are deposited mainly in vital organs, leading to organ dysfunction and subsequently to death; approximately 42% of affected RA subjects die within four years of diagnosis (Cunnane, G. and A. Whitehead (1999) Bailliere's Clin Rheumatol 13:615).

Multiple organ amyloidosis is asymptomatic over an extended period of time and is largely undiagnosed. Little is known about the duration of the sub-clinical phase, but it can be protracted and, therefore, clinical diagnosis of MOA e.g., AA amyloidosis and AL amyloidosis, is often delayed or missed until the amyloid deposits are extensive. For example, overall, diagnosed cases of AA amyloidosis represent only 17% of total actual cases, i.e., 83% of all suspected cases of AA amyloidosis are undiagnosed. Differences in diagnostic techniques (e.g., urinalysis), physician's awareness of MOA, e.g., AA amyloidosis and AL amyloidosis, and willingness to proceed with a biopsy, all contribute to the under-diagnosis of the disease. Even when symptomatic, only 53% of cases are diagnosed. By the time a diagnosis of MOA is made, many of the subjects have already progressed significantly and have end-stage renal disease (ESRD). Early diagnosis of MOA remains a vital treatment objective for the successful management of the disease and for the preservation of subjects' quality of life.

No blood test is available for the diagnosis of MOA, e.g., AA Amyloidosis or AL amyloidosis. Although the plasma concentrations of SAA and C-reactive protein are usually elevated in subjects with AA amyloidosis when the disorder presents, these are non-specific biomarkers of inflammatory conditions (Cunnane, G. and A. Whitehead (1999) Bailliere's Clin Rheumatol 13:615). Diagnosis of MOA, e.g., AA amyloidosis and AL amyloidosis, is accomplished through tissue biopsy (renal, rectal, gastric, gingival, fat, salivary labial glands) and histology. Renal biopsy is necessary for a definitive diagnosis, but if deemed undesirable or inappropriate, diagnosis by fat aspiration can reveal MOA, e.g., AA amyloidosis and AL amyloidosis in about 35-84% of subjects, and rectal biopsy can confirm the presence of amyloid, e.g., AA amyloid and AL amyloid in up to 69-97% of cases (Gertz, M. A. and R. A. Kyle (1991) Medicine (Baltimore) 70:246; Hachulla, E. and Grateau, G. (2002) Joint Bone Spine 69:538; Kuroda, T., et al., (2002) Clin Rheumatol 21:123; Libbey, C. A., et al., (1983)Arch Intern Med 143:1549; Westermark P, Stenkvist B. (1973) Arch Intern Med. 132(4):522; Yamada, M., et al., (1985) Hum Pathol 16:1206). Amyloid deposits are identified based on their ability to produce characteristic apple-green birefringence under specific polarized light when stained with Congo red. Confirmation of AA-type fibrils or AL-type fibrils is achieved immunohistochemically, using specific antibodies to AA or AL protein. In addition, radiolabelled serum amyloid P (SAP) scanning (¹²³I-SAP scintigraphy) can diagnose the presence of amyloid and can be used to quantitatively monitor the accumulation of the deposits, but it is only available in a few specialized centers in Europe.

Treatment of MOA, e.g., AA Amyloidosis and AL amyloidosis, is a vital unmet medical need. Currently there is no specific therapy for MOA, e.g., AA Amyloidosis or AL amyloidosis and treatment is primarily limited to managing the underlying inflammatory disease or cancer and supporting declining organ function, e.g., dialysis (Falk, R., et al., (1997) N Eng J Med 337:898). Once amyloid deposits have formed, there is no known, widely accepted therapy or treatment which significantly dissolves amyloid deposits in situ or that prevents further amyloid deposition.

While no current treatment is completely effective, there are promising treatments under development. Treatments may therefore become available in the near future, however, it is still necessary to diagnose patients as early as possible in the progression of MOA to increase the benefits of treatment.

Moreover, since it is difficult to diagnose MOA because there are few specific symptoms, biopsy is necessary to make a definitive diagnosis of this disease. However, biopsy is an invasive procedure associated with significant risks. Therefore, it would be very useful if diagnosis of MOA, e.g., AA amyloidosis and/or AL amyloidosis, was possible by a convenient, noninvasive technique such as blood test or imaging method.

The present invention provides convenient, non-invasive diagnostic methods and kits, comprising materials for use in such methods, for the diagnosis of multiple organ amyloidosis (MOA), e.g., AA amyloidosis or AL amyloidosis. The invention generally is directed to the detection of two or more biomarkers associated with MOA, e.g., AA Amyloidosis or AL amyloidosis. The present invention further provides a means of monitoring the regression, progression or treatment of MOA, e.g., AA amyloidosis or AL amyloidosis and assessment of therapeutic agents and/or regimens.

### DETAILED DESCRIPTION OF THE INVENTION

As used herein, “multiple organ amyloidosis” or “MOA” includes amyloidoses, such as for example, AA amyloidosis and AL amyloidosis, that affect multiple organs. Multiple organ amyloidosis (MOA) is often associated with an underlying condition that leads to the deposition of fibrils in different organs (for example, spleen, liver, heart, kidney, intestinal wall). Subjects at risk of developing MOA, e.g., AA amyloidosis or AL amyloidosis, are typically afflicted with chronic inflammatory or infectious diseases or have multiple myeloma or a plasma cell dyscrasia. Those subjects who do not respond well to chemotherapy or anti-inflammatory therapies and do not control their inflammatory response have a higher risk of developing MOA than subjects who respond well to chemotherapy or anti-inflammatory therapies. Several inflammatory mediators, e.g., biomarkers of MOA, e.g., AA amyloidosis or AL amyloidosis, have been found to be modulated, e.g., aberrant, in subjects with MOA, e.g., a biomarker-associated disease. However, each of these biomarkers, when considered individually, is not necessarily a reliable indicator of the presence of a biomarker-associated disease, e.g., MOA, e.g., AA amyloidosis or AL amyloidosis, in subjects with active chronic inflammatory or infectious conditions or a plasma cell dyscrasia or multiple myeloma. It has been discovered that consideration of such biomarkers as a group, as described in the present invention provides a reliable diagnosis of MOA, e.g., AA amyloidosis or AL amyloidosis, or the risk of developing MOA, e.g., AA amyloidosis or AL amyloidosis.

The inflammatory mediators found to be aberrant, e.g., abnormally high in subjects with AA amyloidosis are, SAA, IL-18, M-CSF, and HGF. Each of these biomarkers has been found to reach levels in AA amyloidosis subjects which exceed those found in healthy individuals as well as in individuals with chronic inflammatory conditions that are well controlled with medication. The inflammatory mediator found to be aberrant, e.g., abnormally high in subjects with AL amyloidosis is HGF. This biomarker has been found to reach levels in AL amyloidosis subjects which exceed those found in healthy individuals as well as in individuals with chronic inflammatory conditions that are well controlled with medication.

Additional biomarkers have been identified based on their modulation in subjects with chronic inflammatory conditions that are well controlled with medication and in subjects with AA amyloidosis or AL amyloidosis. It was found that subjects with AA amyloidosis have autoantibodies against citrulinated vimentin (Sa) and show a marked decrease in urine glycosaminoglycan (GAG) concentrations. In addition, variant alleles of SAA, e.g., 1.3 and 1.5, have been associated with higher levels of SAA in RA subjects that renders them more susceptible to amyloidosis. In subjects with AL amyloidosis, a marked decrease in urine glycosaminoglycan (GAGs) concentration, an increase in urine albumin, and an increase in creatinine clearance were observed as was the presence of monoclonal immunoglobulin L chain.

When considered individually, these biomarkers do not necessarily indicate the presence of MOA, e.g., AA amyloidosis or AL amyloidosis in subjects that are asymptomatic or undiagnosed with MOA, e.g., AA amyloidosis or AL amyloidosis. The present invention provides methods and kits for diagnosis, prognosis and treatment assessment in which two or more biomarkers of the invention are utilized to identify subjects early in the development of MOA, e.g., AA amyloidosis or AL amyloidosis, particularly those that do not present with all the clinical hallmarks of the disease typically utilized to diagnose the disease. The present invention, therefore, substantially advances the state of the art with respect to a non-invasive method to diagnose MOA, e.g., AA amyloidosis or AL amyloidosis prior to significant organ damage.

One of the many advantages of the present invention is that the simultaneous evaluation of the biomarkers of the invention in, e.g., subjects with chronic inflammation or infectious diseases or in subjects genetically predisposed to the development of AA amyloidosis (e.g., Familial Mediterranean Fever (FMF), Rheumatoid Arthritis) or in subjects with a plasma cell dyscrasia or multiple myeloma may lead to the identification of subjects who either are at a higher risk of developing MOA or have early MOA which is still silent or undiagnosed.

These subjects would benefit from having a test aimed at detecting the presence of MOA, e.g., AA amyloidosis or AL amyloidosis, as early as possible so that disease progression can be retarded or even halted. Optionally, subjects with a specific biomarker profile may then be biopsied to confirm the presence of MOA.

Accordingly, the invention relates to newly discovered correlations between the presence, absence, and level of certain biomarkers, e.g., MOA biomarkers, e.g., AA amyloidosis or AL amyloidosis, biomarkers, and the presence of or risk of MOA, e.g., AA amyloidosis or AL amyloidosis, in a subject. It has been discovered that the presence, absence, and/or level of a combination of biomarkers, e.g., at least two of the biomarkers, described herein correlates with the presence of MOA, e.g., AA amyloidosis or AL amyloidosis, in a subject. Methods are provided for detecting the presence of MOA, e.g., AA amyloidosis or AL amyloidosis, in a subject, the absence of MOA, e.g., AA amyloidosis or AL amyloidosis, in a subject, and other characteristics of MOA, e.g., AA amyloidosis or AL amyloidosis, that are relevant to prevention, diagnosis, characterization, and therapy of MOA, e.g., AA amyloidosis or AL amyloidosis, in a subject.

Various aspects of the invention are described in further detail in the following subsections:

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

A “biomarker” refers to any biologically-based marker of a condition. A “multiple organ amyloidosis biomarker” or a “MOA biomarker”, refers to a biologically-based marker of multiple organ amyloidosis; an “AA amyloidosis biomarker” or an “AL amyloidosis biomarker”, refers to a biologically-based marker of AA amyloidosis or AL amyloidosis, respectively. Biomarkers include, but are not limited to alleles, genes, peptides, antibodies (e.g., an autoantibody), or other biologics for which the presence, absence, or altered level of such biomarker is associated with a disease state (e.g., multiple organ amyloidosis (MOA), e.g., AA amyloidosis or AL amyloidosis), as compared to a suitable control (e.g., the presence or level of the biologic in a normal or healthy sample). Suitable biologic samples also include blood samples, e.g., such as a plasma and/or serum, urine, stool, cerebrospinal (i.e. CSF) and spinal fluid, synovial fluid, conjunctival fluid, salivary fluid, lymph, bile, tears, and sweat), tissues and cells, e.g., monocytes and epithelial cells, e.g., gingival epithelial cells.

A “biomarker allele” is an allele corresponding to a biomarker of the invention.

A “biomarker nucleic acid” is a nucleic acid (e.g., genomic DNA, mRNA, cDNA) encoded by or corresponding to a biomarker of the invention. The biomarker nucleic acids also include RNA comprising the entire or a partial sequence of any biomarker of the invention, wherein all thymidine residues are replaced with uridine residues. A “biomarker protein” is a protein encoded by or corresponding to a biomarker of the invention. A biomarker protein comprises the entire or a portion of a biomarker of the invention. The terms “protein,” “peptide” and “polypeptide’ are used interchangeably. A “biomarker antibody” or “biomarker autoantibody”, as used interchangeably herein, is intended to include immunoglobulin molecules and immunologically active portions of immunoglobulin molecules, i.e., molecules that contain at least one antigen binding site which specifically binds (immunoreacts with) an antigen, such as Fab and F(ab′)₂ fragments.

The term “allele” refers to alternative forms of a gene or portions thereof. Alleles occupy the same locus or position on homologous chromosomes. When a diploid subject has two identical alleles of a gene, the subject is said to be homozygous for the gene or allele, e.g., have a homozygous genotype at that allele. When a subject has two different alleles of a gene, the subject is said to be heterozygous for the gene or allele, e.g., have a heterozygous genotype at that allele. Alleles of a specific gene can differ from each other at a single nucleotide, or several nucleotides, and said differences can include substitutions, deletions, and insertions of nucleotides. An allele of a gene can also be a form of a gene containing one or more mutations as compared to the spectrum of known alleles present in a population.

The term “allelic variant of a polymorphic region of a gene” or “allelic variant”, used interchangeably herein, refers to an alternative form of a gene having one of several possible nucleotide sequences found in that region of the gene in the population. As used herein, allelic variant is meant to encompass functional allelic variants, non-functional allelic variants, SNPs, mutations and polymorphisms.

A “polymorphic gene” refers to a gene having at least one polymorphic region.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic locus can contain a single variant nucleotide, the identity of which differs in the other alleles found in the population. A polymorphic locus can also contain variants of more than one nucleotide in length, e.g., insertions, deletions, multiple SNPs.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a single nucleotide or an insertion of a single nucleotide relative to a reference allele. Typically the polymorphic site is occupied by a base other than the reference base. For example, where the reference allele contains the base “T” (thymine) at the polymorphic site, the altered allele can contain a “C” (cytosine), “G” (guanine), or “A” (adenine) at the polymorphic site. SNPs may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing or misregulation of transcription, or it may have no effect.

A “diagnostically predictive collection of biomarkers” e.g., a “multiple organ amyloidosis diagnostically predictive collection of biomarkers”, a “MOA diagnostically predictive collection of biomarkers”, an “AA amyloidosis diagnostically predictive collection of biomarkers” or an “AL amyloidosis diagnostically predictive collection of biomarkers”, refers to the number of biomarkers sufficient to diagnose or prognose a particular MOA in a targeted subject and/or population. The number of biomarkers sufficient to diagnose or prognose a particular MOA will vary based on, for example, the type of MOA, the race and/or ethnicity of a subject and/or population, the stage of the disease at the time of diagnosis, age of a subject, sex of a subject, clinical evaluation of a subject, etc.

The terms “therapeutic agent” or “drug” refer to an agent having a beneficial ameliorative or prophylactic effect on a specific disease or condition in a living human or non-human animal. In the case of MOA, e.g., AA or AL amyloidosis, the agent may, for example, improve or stabilize specific organ function or may inhibit or reduce amyloid deposition. As an example, renal function may be stabilized or improved by 10% or greater, 20% or greater, 30% or greater, 40% or greater, 50% or greater, 60% or greater, 70% or greater, 80% or greater, or by greater than 90%. Suitable therapeutic agents that can be employed in the methods, compositions or kits of the present invention include the compounds disclosed in International Patent Publication Nos. WO 94/22437 and WO 96/28187, both entitled “Methods for Treating Amyloidosis” and WO 00/64420, entitled “Compositions and Methods for Treating Amyloidosis,” the entire contents of which are incorporated herein in their entirety by this reference. In one embodiment, the therapeutic agent employed is propane-1,3-disulfonic acid and its pharmaceutically acceptable equivalents, including but not limited to, disodium propane-1,3-disulfonate. Therapeutic agents further include any other agents that can be employed to treat MOA, e.g., AA or AL amyloidosis, including those described in Pepys, et al., Targeted Pharmacological Depletion of Serum Amyloid P Component for Treatment of Human Amyloidosis, Nature 417:254-59 (May 16, 2002), the entire contents of which is incorporated herein in its entirety by this reference, e.g., the compound R-1 [6-[R-2-carboxy-pyrrolidin-1-yl]-6-oxo-hexanoyl]pyrrolidine-2-carboxylic acid (CPHPC).

“Modulation” of amyloid (e.g., amyloid AA or AL protein) deposition includes both inhibition, as defined herein, and enhancement of amyloid deposition or fibril formation. The term “modulating” is intended, therefore, to encompass prevention or cessation of amyloid formation or accumulation, inhibition or slowing down of further amyloid formation or accumulation in a subject with ongoing MOA, e.g., already having amyloid deposition, and reducing or reversing of amyloid formation or accumulation in a subject with ongoing amyloidosis; and enhancing amyloid deposition, e.g., increasing the rate or amount of amyloid deposition in vivo or in vitro. Amyloid-enhancing compounds may be useful in animal models of MOA, for example, to make possible the development of amyloid deposits in animals in a shorter period of time or to increase amyloid deposits over a selected period of time. Amyloid-enhancing compounds may be useful in screening assays for compounds which inhibit amyloidosis in vivo, for example, in animal models, cellular assays and in vitro assays for MOA. Such compounds may be used, for example, to provide faster or more sensitive assays for compounds. Modulation of amyloid deposition is determined relative to an untreated subject or relative to the treated subject prior to treatment.

“Inhibition” of amyloid deposition includes prevention or cessation of amyloid formation, e.g., fibrillogenesis, clearance of amyloid, for example, but not limited to, soluble amyloid A protein from, e.g., kidney, spleen, liver, inhibiting or slowing down of further amyloid deposition in a subject with MOA, e.g., already having amyloid deposits, and reducing or reversing amyloid fibrillogenesis or deposits in a subject with ongoing MOA. Inhibition of amyloid deposition is determined relative to a suitable control. Exemplary suitable controls include an untreated subject, the treated subject prior to treatment, stabilization of the subject, and clinically measurable improvements.

As used herein, the allele occurring most frequently in a selected population is referred to as the “reference and/or wild-type form or allele”. Other allelic forms are designated as “alternative or variant forms or alleles”. The “presence” of one or more of a variant biomarker allele, e.g., a heterozygous or homozygous genotype at the biomarker allele locus, in a subject, e.g., a human subject or wild-type animal or subject, is intended to mean that the subject is at risk of developing or has developed a disease, such as MOA, e.g., AA amyloidosis. The “absence” of one variant biomarker allele, e.g., a homozygous wild-type genotype at the biomarker allele locus, in a subject is intended to mean that the subject is not at risk of developing or has not developed a disease, such as MOA, e.g., AA amyloidosis.

As used herein, “a subject at risk of developing” or a “subject that has developed” a biomarker associated disease, e.g., MOA, e.g., AA amyloidosis or AL amyloidosis, includes a subject which has been determined to have a higher probability of developing a biomarker associated disease, e.g., MOA, e.g., AA amyloidosis or AL amyloidosis, when compared to an average representative of the population.

As used herein, “a subject not at risk of developing” or a “subject that has not developed” a biomarker associated disease, e.g., MOA, e.g., AA amyloidosis or AL amyloidosis, includes a subject which has been determined to have a lower probability of developing a biomarker associated disease, e.g., MOA, e.g., AA amyloidosis or AL amyloidosis, when compared to a subject that is at risk of developing a biomarker associated disease, e.g., MOA, e.g., AA amyloidosis or AL amyloidosis.

The “normal” level of a biomarker nucleic acid or protein is the level of the biomarker nucleic acid or protein in a subject or a sample from a subject, e.g., blood, e.g., serum or plasma, urine, stool, bile, tissues or cells, of a subject, e.g., a human subject or wild-type animal or subject not at risk of developing or who has not developed MOA, e.g., AA amyloidosis or AL amyloidosis (e.g., sample from a subject not having the biomarker associated disease). A “control” subject or animal typically has normal levels of a biomarker nucleic acid or protein.

“An aberrant level” of a biomarker nucleic acid or protein is any level of a biomarker nucleic acid or protein that differs from the normal level of, e.g., significantly higher or elevated levels, or significantly lower or depressed levels of a biomarker nucleic acid or protein.

A “higher level” or “elevated level” of a biomarker refers to a level that is elevated relative to a suitable control. Preferably, the differential from the suitable control, if any, is greater than the standard error of the assay employed to assess the level. Moreover, the elevated level is preferably at least twice, and more preferably three, four, five or ten times the level of the biomarker in a suitable control (e.g., sample from a subject not having the biomarker associated disease, or the average level of the biomarker nucleic acid or protein in several control samples or other suitable benchmark).

A “depressed level” or “lower level” of a biomarker refers to a level that is depressed relative to a suitable control. Preferably, the differential from the suitable control, if any, is greater than the standard error of the assay employed to assess the level. The depressed level preferably is at least twice, and more preferably three, four, five or ten times lower than the level of the suitable control (e.g., level in a healthy subject not having the biomarker associated disease, e.g., MOA, e.g., AA amyloidosis or AL amyloidosis, or the average level of the biomarker nucleic acid or protein in several control samples or other suitable benchmark).

The term “probe” refers to any molecule which is capable of selectively binding to a specifically intended target molecule, for example, a nucleotide transcript, protein or autoantibody encoded by or corresponding to a biomarker nucleic acid protein or autoantibody. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. In some cases, all nucleotide residue positions of each of the portions are occupied by the same nucleotide residue.

A molecule is “fixed” or “affixed” to a substrate if it is covalently or non-covalently associated with the substrate such that the substrate can be rinsed with a fluid (e.g. standard saline citrate, pH 7.4) without a substantial fraction of the molecule dissociating from the substrate.

Multiple organ amyloidosis, e.g., AA amyloidosis or AL amyloidosis, is “modulated” if at least one symptom of the disease is increased or exacerbated, or inhibited, e.g., alleviated, terminated, slowed, or prevented. As used herein, MOA, e.g., AA amyloidosis or AL amyloidosis is also “modulated” if recurrence of the disease is increased or exacerbated or inhibited, e.g., alleviated, terminated, slowed, or prevented.

The terms “cure,” “heal,” “alleviate,” “relieve,” “alter,” “remedy,” “ameliorate,” “improve” and “affect” are evaluated in terms of a suitable or appropriate control. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to the determination that a subject is at risk of developing or has developed MOA, e.g., AA amyloidosis or AL amyloidosis. In another embodiment, a “suitable control” or “appropriate control” is a value, level, feature, characteristic, property, etc. determined in a subject, e.g., a control or normal subject exhibiting, for example, normal traits. In yet another embodiment, a “suitable control” or “appropriate control” is a predefined value, level, feature, characteristic, property, etc.

A kit is any manufacture (e.g. a package or container) comprising at least one reagent, e.g. a probe, for specifically detecting the presence, absence or level of a biomarker of the invention. The kit may be promoted, distributed, or sold as a unit for performing the methods of the present invention. The reagents included in such a kit may comprise, for example, probes, primers and/or antibodies for use in detecting the biomarkers of the invention. Kits may also contain specific dyes to detect the presence of biomarkers of the invention by spectrophotometry. In addition, the kits of the present invention may contain instructions that describe a suitable detection assay. Such kits can be conveniently used, e.g., in clinical settings, to diagnose subjects that may or may not be exhibiting symptoms of MOA, e.g., AA amyloidosis or AL amyloidosis, or are suspected of being at risk of developing MOA, e.g., AA amyloidosis or AL amyloidosis.

Unless otherwise specified herein, the terms “antibody” and “antibodies” broadly encompass naturally-occurring forms of antibodies (e.g., IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

Amyloidoses

“Amyloidoses” are a group of diseases characterized by extracellular deposition of proteins in characteristic amyloid fibrils. These insoluble fibrillar proteins can be localized in one specific site, such as pancreas or brain (referred to as “localized amyloidosis”), or can be broadly distributed in several vital organs such as kidneys, liver, spleen, and heart (referred to as “systemic amyloidosis”). (Falk, R., et al., (1997) N Engl J Med 337:898). Some amyloid diseases can be idiopathic, but most of these diseases appear as a complication of a previously existing disorder. For example, primary amyloidosis, e.g., AL amyloidosis, can appear without any other pathology or can follow plasma cell dyscrasia or multiple myeloma. Once these amyloids have formed, there is no known, widely accepted therapy or treatment which significantly dissolves amyloid deposits in situ, prevents further amyloid deposition or prevents the initiation of amyloid deposition. Ultimately, amyloid deposition leads to organ dysfunction, organ failure, and eventually death (Cunnane, G. (2001) Lancet 358:24). During fibril formation, elements such as glycosaminoglycans (GAGs) interact with the amyloid protein, promote a structural shift process, and favor the deposition of fibrils in the organs.

Historically, amyloidoses were classified as either systemic or localized diseases. Systemic amyloidoses include primary, secondary/reactive, and hereditary or familial amyloidosis, while localized amyloidoses include several diseases with specific targeted organs. The modern classification of amyloidosis is based on the nature of the fibrils, e.g., the type of protein deposited in the fibrils, (Table 1) (Sipe, J. and Cohen, A. (2000) J. Struct Biol 130:88; Husby, G., et al., (1994) Clin Immunol Immunopathol 70:2; Westermark, P., et al., (2002) Amyloid 9:197). Primary and secondary amyloidosis that affect more than one organ, e.g., form deposits in more than one organ and/or affect the function of more than one organ, is referred to herein as multiple organ amyloidosis or MOA.

“AL amyloidosis”, also called “Primary amyloidosis” results in immunoglobulin light chain amyloid deposition that is generally associated with almost any dyscrasia of the B lymphocyte lineage, ranging from malignancy of plasma cells (multiple myeloma) to benign monoclonal gammopathy. At times, the presence of amyloid deposits may be a primary indicator of the underlying dyscrasia. AL amyloidosis is also described in detail in Current Drug Targets, 2004, 5 159-171.

Fibrils of AL amyloid deposits are composed of monoclonal immunoglobulin light chains or fragments thereof. More specifically, the fragments are derived from the N-terminal region of the light chain (kappa or lambda) and contain all or part of the variable (V_{L}) domain thereof. Deposits generally occur in the mesenchymal tissues, causing peripheral and autonomic neuropathy, carpal tunnel syndrome, macroglossia, restrictive cardiomyopathy, arthropathy of large joints, immune dyscrasias, myelomas, as well as occult dyscrasias. However, it should be noted that almost any tissue, particularly visceral organs such as the liver, spleen, heart, and especially the kidney, may be involved. Examples of AL proteins include, but are not limited to immunoglobulin lambda (λ) chains such as λ VI (λ6 chains), or λ III chains, and (κ) chains.

There are also many forms of MOA that are hereditary. Although they are relatively rare conditions, adult onset of symptoms and their inheritance patterns (usually autosomal dominant) lead to persistence of such disorders in the general population. Generally, the syndromes are attributable to point mutations in the precursor protein leading to production of variant amyloidogenic peptides or proteins.

“AA Amyloidosis” also called “Secondary Amyloidosis” or “Reactive Amyloidosis”, was so named because it develops secondarily to chronic inflammatory conditions, such as rheumatoid arthritis (RA) or Familial Mediterranean Fever. It is now most often called AA Amyloidosis, since a major factor in the protein deposition process involves the precursor protein AA, a cleaved product of the acute phase protein serum amyloid A (SAA) (Cunnane, G. (2001) Lancet 358:24; Sipe, J. and Cohen, A. (2000) J Struct Biol 130:88; Cunnane, G. and Whitehead, A. (1999) Bailliere's Clin Rheumatol 13:615).

AA fibrils are generally composed of 8,000 Dalton fragments (AA peptide or protein) formed by proteolytic cleavage of SAA, a circulating apolipoprotein that is mainly synthesized in hepatocytes in response to such cytokines as IL-1, IL-6 and TNF. Once secreted, SAA is complexed with HDL. Deposition of AA fibrils can be widespread in the body, with a preference for parenchymal organs. The kidneys are usually a deposition site, and the liver and the spleen may also be affected. Deposition is also seen in the heart, gastrointestinal tract, and the skin.

Underlying diseases which can lead to the development of AA amyloidosis include, but are not limited to, inflammatory diseases, such as rheumatoid arthritis, juvenile chronic arthritis, ankylosing spondylitis, psoriasis, psoriatic arthropathy, Reiter's syndrome, Adult Still's disease, Behcet's syndrome, and Crohn's disease. AA deposits are also produced as a result of chronic microbial infections, such as leprosy, tuberculosis, bronchiectasis, decubitus ulcers, chronic pyelonephritis, osteomyelitis, and Whipple's disease. Certain malignant neoplasms can also result in AA fibril amyloid deposits. These include such conditions as Hodgkin's lymphoma, renal carcinoma, carcinomas of gut, lung and urogenital tract, basal cell carcinoma, and hairy cell leukemia. Other underlying conditions that may be associated with AA amyloidosis are Castleman's disease and Schnitzler's syndrome.

TABLE 1
Classification of Exemplary Amyloidoses

| Amyloid | | Systemic/ | |
|---|---|---|---|
| Protein | Protein Precursor | Localized | Related Disease |
| AA | Serum Amyloid A | S | Chronic inflammation or |
| | | | infection |
| AL | λ or κ light chains | S, L | Plasma cell clone related |
| ATTR | Transthyretin (TTR) | S | Familial, related to TTR |
| | | | mutations |
| | | | Old age, related to wild- |
| | | | type TTR |
| Aβ | Aβ protein precursor | L | Alzheimer's disease |
| | | | Cerebral amyloid |
| | | | angiopathy |
| Aβ₂M | Beta₂-microglobulin | S | Chronic hemodialysis |

Pathogenesis of AA Amyloidosis

1. Development of AA Amyloidosis

Chronic inflammation or infection that gives rise to an acute-phase protein response is a prerequisite to the development of AA Amyloidosis. The concentration of serum amyloid A protein (SAA), the circulating precursor of AA amyloid fibrils, increases substantially in response to many types of inflammation (Cunnane, G. (2001) Lancet 358:24; Cunnane, G. and Whitehead, A. (1999) Bailliere's Clin Rheumatol 13:615; Kisilevsky, R., et al. (1995) Nat Med. 1(2):143). For reasons that remain unclear, in a proportion of subjects whose levels remain elevated for longer periods, a cleavage fragment of SAA aggregates and is deposited in the tissues as AA amyloid fibrils.

2. AA Amyloid Fibril

Amyloid deposits are complex structures where the constituent amyloid fibrils are intertwined with other proteins including basement membrane components, serum amyloid P component (SAP), complement components, ApoE, and heparan sulfate proteoglycan (HSPG) (Cunnane, G. (2001) Lancet 358:24; Sipe, J. and Cohen, A. (2000) J. Struct Biol 130:88; Husby, G., et al., (1994) Clin Immunol Immunopathol 70:2; Cunnane, G. and Whitehead, A. (1999) Bailliere's Clin Rheumatol 13:615; Kisilevsky, R., et al. (1995) Nat Med. 1(2):143). HSPGs are common constituents of amyloid deposits in all known types of amyloidosis thus far investigated and are thought to play an active role in the initiation of fibril formation and deposition. Through its binding to amyloidogenic proteins, the highly sulfated glycosaminoglycan (GAG) moiety portion of proteoglycans promotes fibril formation.

3. The AA ‘Amyloid Cascade’

Through binding of the amyloid proteins, GAGs promote a structural shift of the amyloid, which then becomes fibrillar. The fibrils deposit around cells in affected organs. Amyloid fibril production and decreased clearance of amyloid protein result in a cascade of events leading to disruption of overall organ function. For example, the formation and deposition of fibrils in the kidney are key players in the progression of renal impairment to end-stage renal disease (ESRD).

Risk Factors for AA Amyloidosis

The most common underlying disorders in industrialized countries are chronic idiopathic inflammatory rheumatic diseases such as rheumatoid arthritis (RA) (Table 2). The frequency with which AA Amyloidosis occurs in subjects with chronic idiopathic inflammatory rheumatic diseases varies according to the specific inflammatory disease, the subject population, and the method of diagnosis. About 3-10% of subjects with RA will develop AA Amyloidosis. In the US, RA is responsible for almost half of all cases (L.E.K. Consulting. Independent Market Research. 2003). Although chronic inflammation is a prerequisite to the development of AA Amyloidosis, the various factors that determine susceptibility to the disorder, and those that influence its clinical course and outcome, are not well known (Cunnane, G. (2001) Lancet 358:24; Cunnane, G. and Whitehead, A. (1999) Bailliere's Clin Rheumatol 13:615; Joss, N., et al (2000) QJM 93:535; Mimouni, A., et al., (2000) Pediatrics 105:E70).

TABLE 2
Lifetime Incidence of AA Amyloidosis in
Subjects With Inflammatory Conditions

Incidence of AA Amyloidosis during the course of the
underlying disorder

| | |
|---|---|
| Rheumatoid Arthritis | 3-10% |
| Psoriatic Arthritis | 3-13% |
| Chronic Juvenile Arthritis | 0.14-17% |
| Ankylosing Spondylitis in children | 4-5% |
| Inflammatory Bowel Disease | 0.4-2% |
| Familial Mediterranean Fever | 8-37% |
| Chronic Infectious Diseases (tuberculosis, leprosy, | Up to 10% |
| bronchiectasis, chronic osteomyelitis, chronic | |
| pyelonephritis) | |

Table derived from: Cunnane, G. and Whitehead, A. (1999) Bailliere's Clin Rheumatol 13: 615; Buxbaum, J. www.emedicine.com/med/topic 3388.htm; updated 2002; 1-17; Rocken, C. and Sletter, K. (2003) Virchows Arch 443: 3.

Diagnosis of AA Amyloidosis

The most critical factor in diagnosing AA Amyloidosis is first considering the possibility that it could be present. AA Amyloidosis should be suspected in any subject with longstanding inflammatory disease, especially if poorly controlled. Renal dysfunction is the most common presentation of AA Amyloidosis. The development of persistent proteinuria, elevated serum creatinine, or chronically elevated serum amyloid A (SAA), C-reactive protein (CRP), and erythrocyte sedimentation rate (ESR) in any individual with a chronic inflammatory disease should prompt the physician to consider AA Amyloidosis and evaluate the subject further. Subjects may also present with a variety of gastrointestinal complaints.

Pathogenesis of AL Amyloidosis

1. Development of AL Amyloidosis

Systemic AL amyloidosis, formerly known as primary amyloidosis, is a disorder associated with a clonal plasma cell dyscrasia or multiple myeloma (Falk, et al, (1997) NEJM 337:898-912). Multiple organ disease results from the extracellular deposition of monoclonal immunoglobulin light chain fragments in an abnormal insoluble fibrillar form. Amyloid fibrils associate in vivo with the normal plasma protein serum amyloid P component (SAP), and this phenomenon is the basis for the use of SAP scintigraphy for imaging and monitoring amyloid deposits (Hawkins, et al, (1990) NEJM 323:508-513). Accumulation of amyloid progressively disrupts the normal tissue structure and ultimately leads to organ failure, frequently including the kidneys, heart, liver and peripheral nervous system (Kyle & Gertz, (1995) Seminars in Hematology 32:45-59).

AL amyloidosis may be associated with myeloma or other B-cell malignancy, but in most cases the underlying plasma cell dyscrasia is subtle and non-proliferating, analogous to monoclonal gammopathy of undetermined significance (MGUS).

2. AL Amyloid Fibril

AL amyloid fibrils are derived from the N-terminal region of monoclonal immunoglobulin light chains and consist of the whole or part of the variable (V_{L}) domain. Intact light chains may rarely be found, and the molecular weight therefore varies between about 8000 and 30,000 Da. All monoclonal light chains are unique and the propensity for certain ones to form amyloid fibrils is an inherent property related to their particular structure. Monoclonal light chains that can form amyloid are able to exist in partly unfolded states, involving loss of tertiary or higher order structure. These readily aggregate with retention of β-sheet secondary structure into protofilaments and fibrils.

3. The AL ‘Amyloid Cascade’

As discussed above for AA amyloidosis, HSPGs are common constituents of amyloid deposits in all known types of amyloidosis and the binding of GAGs to amyloidogenic proteins promotes fibril formation. Subsequently, GAGs promote a structural shift of the amyloid, which then becomes fibrillar. The fibrils deposit around cells in affected organs. Amyloid fibril production and decreased clearance of amyloid protein result in a cascade of events leading to disruption of overall organ function.

Once the process of protofilament and fibril formation has started, ‘seeding’ may also play an important facilitating role, so that amyloid deposition may progress exponentially as expansion of the amyloid template ‘captures’ further precursor molecules. Only a small proportion of monoclonal light chains are amyloidogenic, but it is not possible to identify these from their class or abundance.

Diagnosis of AL Amyloidosis

Most symptoms of AL amyloidosis are nonspecific and are not initially recognized. Weight loss, weakness and fatigue, followed by pupura are the most common symptoms. As with AA amyloidosis, renal dysfunction is the most common presentation of AL Amyloidosis. The development of persistent proteinuria, decrease in serum immunoglobulin and the presence of monoclonal immunoglobulin L chain in the serum or urine should prompt the physician to consider AL Amyloidosis and evaluate the subject further. Subjects may also present with heart palpitations or lightheadedness, dysesthesia and a variety of gastrointestinal complaints. In addition, any evidence of a plasma cell dyscrasia should be considered supportive evidence for a diagnosis of AL amyloidosis.

The standard treatment of AL amyloidosis aims to reduce production of the monoclonal immunoglobulin precursor via chemotherapy or occasionally via radiation therapy or surgery of a localized amyloidogenic plasmacytoma, however if significant organ damage has occurred it may not be reversible.

Exemplary Biomarkers of the Invention

The diagnosis of amyloidosis currently relies on biopsy and the pathological demonstration of typical amyloid deposits (Shikano, Kushimoto, et al., 2000). Since biopsy is an invasive examination, a non-invasive and sensitive test for MOA, e.g., AA amyloidosis or AL amyloidosis is needed. Rapid, simple, and cheap techniques in diagnosis of MOA, e.g., AA amyloidosis or AL amyloidosis, would promote early treatment and thus improve prognosis of the disease. Ideal biomarkers of the invention for the diagnosis, prognosis and assessment of treatment of a biomarker associated disease, MOA, e.g., AA amyloidosis or AL amyloidosis, are those that are specific, sensitive, reproducible, noninvasive, and not influenced by age or sex, and accurately reflect the disease process, have a clear normal range, and change, e.g., modulate, during the progression of the disease. The present invention has thus identified that measurement of a combination of biomarkers described below, e.g., at least two, provide specific, sensitive, and reproducible methods of diagnosing, prognosing and assessing treatment of MOA, e.g., AA amyloidosis or AL amyloidosis.

Serum Amyloid A (SAA) and C-Reactive Protein (CRP)

The term “acute phase response” refers to a number of very complex endocrine and metabolic or neurological changes observed in an organism, either locally or systemically, a short time after injuries or the onset of infections, immunological reactions, and inflammatory processes. Measurement of the acute phase response or reaction is widely used as an indicator of severity of disease, e.g., a biomarker associated disease such as MOA, e.g., AA amyloidosis, and response to treatment.

Two proteins produced during the acute phase reaction are C-reactive protein (CRP) and serum amyloid A (SAA), both of which have been postulated to be biomarkers useful for the diagnosis and/or prognosis of MOA, e.g., AA amyloidosis (Kumon, Y., et al. (1999) J Rheumatol. 26:785; Cunnane, G. and Whitehead, A. S. (1999) Baillieres Best Pract Res Clin Rheumatol 13:615; Cunnane, G., et al., (2000) J Rheumatol. 27:58).

SAA has been shown to participate in chemotaxis, cellular adhesion, cytokine production, and metalloproteinase secretion and is thus integrally involved in the disease process. In addition to its production by the liver as part of the acute phase response, SAA is also expressed by several pathologic tissues such atherosclerotic plaques and, rheumatoid synovitis. Its constitutive production in normal tissue suggests a role for SAA in host defense and tissue turnover.

C-reactive protein is a protein of the pentraxin family, produced by the liver during periods of inflammation and detectable in serum in various disease conditions particularly during the acute phase of immune response. Normally C-reactive protein is negative in the bloodstream. CRP has been shown to participate in reaction with cell surface receptors resulting in opsonization, enhanced phagocytosis, and passive protection; activation of the classical complement pathway; scavenging for chromatin fragments; inhibition of growth and/or metastases of tumor cells; and modulation of polymorphonuclear function.

Many studies have suggested that SAA is a sensitive biomarker of MOA, e.g., AA amyloidosis disease activity. CRP and SAA usually respond (increase) in parallel, however SAA often achieves much higher levels than CRP. In some studies, SAA was found to increase in accordance with the degree of inflammation, while CRP remained normal (Cunnane, G., et al., (2000) J Rheumatol. 27:58). SAA levels can accurately diagnose low levels of inflammation, however since SAA is a nonspecific biomarker of inflammation it must be interpreted in the context of the full clinical picture.

Nonetheless, early diagnosis of RA may be made in subjects presenting with synovitis and very high SAA levels (Cunnane, G., et al., (2000) J Rheumatol. 27:58). SAA was found to be elevated in subjects presenting for the first time with early inflammatory arthritis. Very high levels of SAA distinguished RA from other forms of arthropathy. Increased concentrations of SAA have also been demonstrated in several chronic inflammatory diseases that may predispose subjects to amyloidosis such as RA, juvenile chronic arthritis, psoriatic arthritis (PsA), etc. Furthermore, in RA, an increased circulating level of SAA is a predisposing condition for amyloid formation (Lange, U., et al., (2000) Rheumatol Int. 19:119).

Concentrations of serum SAA and CRP can be determined by latex agglutination turbidometric immunoassay. SAA can also be measured by ELISA, Western blot analysis or Surface-Enhanced Laser Desorption/Ionization Time-Of-Flight (SELDI-TOF).

Normal SAA levels in healthy subjects are less than about 10 mg/L. Abnormally elevated levels are those equal to or greater than about 10-30 mg/L and greater than 30 mg/L.

Accordingly, suitable biomarkers of MOA, e.g., AA amyloidosis, which can be employed in accordance with the methods and kits of the invention include an elevated SAA level and an elevated CRP level, which can be employed singly or in combination.

SAA Alleles

Serum amyloid A proteins (SAA) is the collective name given to a family of proteins encoded by multiple genes. In humans, two acute phase SAA (A-SAA) genes (SAA1 and SAA2) have been described. A third gene, SAA3, is a pseudogene and SAA4 encodes a constitutive SAA (C-SAA) which is minimally induced during inflammation (Kumon, Y., et al. (1999) J Rheumatol.; 26:785; Cunnane, G., et al., (2000) J Rheumatol. 27:58; Yamada, T., et al., (2001) Ann Rheum Dis. 60:124). Six allelic variants of SAA1 and two allelic variants of SAA2 have been identified.

In a Japanese population, the frequencies of the 1.1, 1.3, and 1.5 variant alleles are equal, whereas the 1.2 variant allele is rare (1-2%). The alleles 1.1 and 1.3 have been considered as positive risk factors of developing AA amyloidosis in white and Japanese subjects, respectively (Yamada, T., et al., (2001) Ann Rheum Dis. 60:124).

The genotype at SAA1 locus did not exhibit a significant correlation with the severity of the renal disorder of AA amyloidosis. Although the mechanism underlying the association of homozygosity for SAA1.3 allele with AA-amyloidosis is still unknown, subjects homozygous for SAA1.3 may have higher levels of SAA proteins than the subjects with other genotypes. As evidence for this hypothesis, a significant positive correlation between the number of SAA1.3 alleles and the mean CRP levels among the 45 subjects with AA-amyloidosis has been demonstrated (Moriguchi, M., et al., (2001) Amyloid. 8:115).

Baba, S., et al. ((1995) Hum Mol Genet. 4:1083) describes an allelic variant of the SAA1 gene that they proposed may represent an important risk factor for the development of reactive amyloid systemic amyloidosis, also called AA amyloidosis. The variant, termed SAA1-gamma, was found in pooled acute-phase serum, using mass spectrometry. SAA1-gamma has alanines at amino acid positions 52 and 57, whereas the previously known SAA1 variants, SAA1-alpha and SAA1-beta, have a valine at position 52 or 57, respectively, instead of an alanine. These SAA1s are the 3 major isoforms of human SAA1. Baba, S., et al. ((1995) Hum Mol Genet. 4:1083) found that SAA1-gamma differed from SAA1-alpha at only 1 base; codon 52 was GCC (ala) in SAA1-gamma and GTC (val) in SAA1-alpha. They found a difference in the distribution of SAA1 genotypes with an increased frequency of gamma/gamma homozygotes in the AA-amyloid group (0.60 vs 0.18).

It was postulated that the allele SAA1γ renders a RA subject susceptible to amyloidosis, possibly by affecting the severity of inflammation in RA. Differences in the susceptibility to AA amyloidosis in animal models have been explained by various genetic and environmental factors associated with the synthesis of SAA, the activity of macrophages in processing SAA to AA, an amyloid-enhancing factor, etc. Japanese RA subjects with the γ/γ genotype have a higher risk of amyloidosis compared with average RA subjects. It has been shown that the γ/γ genotype is a risk factor for amyloidosis but not for RA (Moriguchi, M., et al., (2001) Amyloid. 8:115).

Accordingly, suitable biomarkers of MOA, e.g., AA amyloidosis, that can be employed in accordance with the methods and kits of the invention include the presence of at least one variant allele of SAA, e.g., a heterozygous or homozygous genotype for at least one of the SAA 1.1, 1.3 and γ variant alleles.

Hepatocyte Growth Factor (HGF)

Hepatocyte growth factor (HGF) acts on a variety of epithelial cells in multiple ways and is predominantly produced by mesenchymal cells and macrophages. In the kidney, HGF stimulates the proliferation of renal epithelial cells, and is a potent renotrophic factor involved in regeneration after acute renal failure.

Serum HGF level was measured in subjects with amyloidosis to investigate its usefulness as a diagnostic biomarker of this disease. Subjects were diagnosed as having amyloidosis by biopsy of the kidney or gastrointestinal tract and Congo red or Dylon staining (Shikano, M. et al., (2000) Intern Med. 39:715). A differential diagnosis of AL amyloidosis and AA amyloidosis was also performed. Since all subjects had obvious renal dysfunction, serum HGF was also measured in subjects with chronic glomerulonephritis (CGN), subjects on hemodialysis, and healthy volunteers. The serum HGF level was measured using an HGF ELISA kit (Otsuka Assay Co., Ltd., Tokyo).

In subjects with AA and AL amyloidosis, serum HGF was significantly increased compared with the level in healthy volunteers, subjects with mild CGN (serum creatinine levels<3.0 mg/ml), subjects with severe CGN (serum creatinine level>3.0 mg/ml), and hemodialysis subjects. The mean HGF level of the amyloidosis subjects was 10-fold higher than in the other groups.

Normal HGF levels in healthy subjects are less than 0.28 ng/ml. Abnormally elevated levels are those equal to or greater than about 0.39 ng/ml. Accordingly, in one embodiment, a biomarker of AA amyloidosis is an elevated HGF level. In another embodiment, a biomarker of AA amyloidosis is an elevated HGF level of equal to or greater than about 0.31, 0.33, 0.35, 0.37, 0.39, 0.41, 0.50, 0.6 or 0.75 ng/l.

There was no significant difference between subjects with AA and AL amyloidosis. The serum HGF level of amyloidosis subjects who died within 1 year of measurements was significantly higher than that of subjects who lived for more than 1 year.

Accordingly, suitable biomarkers of MOA, e.g., AA amyloidosis or AL amyloidosis, that can be employed in accordance with the methods and kits of the invention include an elevated HGF level.

Glycosaminoglycans (GAGs)

As discussed above, protein AA, which is a proteolytic cleavage product of SAA protein, may be deposited as amyloid fibrils in different organs, such as, the kidneys, adrenal glands, spleen and liver. This form of amyloidosis occurs in association with inflammatory, immune, infectious or neoplastic disorders. In AL amyloidosis, immunoglobulin light chains are deposited as amyloid fibrils in organs such as kidney, spleen, liver, and heart. This form of amyloidosis is idiopathic or appears in subjects with multiple myeloma. The protein fibrils in AA and AL amyloid deposits are always intimately associated with GAGs. GAGs occur in low concentration in the serum. In the kidney, the proteins are synthesized by the glomerular endothelial, epithelial, and mesangial cells (Tencer, J., et al., (1997) Nephrol Dial Transplant. 12:1161).

A markedly decreased urinary excretion of GAGs, both in relation to urine creatinine and per functioning glomerular area, in both AA- and AL-amyloidosis compared to subjects with other primary glomerular diseases and to healthy controls has been observed. (Tencer, J., et al., (1997) Nephrol Dial Transplant. 12:1161).

Depressed GAGs concentrations in urine were observed in subjects with AA amyloidosis and subjects with AL amyloidosis as compared to healthy controls and subjects with primary glomerular disease (PGD). The concentration of GAGs was less than 1.5 mg/l in subjects with AL and AA amyloidosis, 30.9 mg/l in healthy subjects, and 15.2 mg/l in PDG subjects. 9/10 of the AA amyloidosis subjects and 4/5 of the AL amyloidosis subjects had urine GAGs concentrations below the detection limit of the assay. These results are independent of subject age, albuminuria, serum creatinine, sex, or plasma concentrations of acute phase proteins. Accordingly GAGs is a useful and independent biomarker of renal amyloidosis. (Tencer, Torffvit et al. 1997).

Accordingly, in one embodiment, a biomarker of MOA, e.g., AA amyloidosis or AL amyloidosis, is a GAGs concentration of less than about 10 mg/l, 5 mg/l, 4 mg/l, 3 mg/l, 2 mg/l, or 1.5 mg/l.

Depressed median urine GAGs to creatinine ratio (GCR), and depressed median fractional GAG excretion (FGE), were also observed. (Tencer, J., et al., (1997) Nephrol Dial Transplant. 12:1161).

Accordingly, suitable biomarkers of MOA, e.g., AA amyloidosis or AL amyloidosis, that can be employed in accordance with the methods and kits of the invention include depressed GCR ratio values and depressed FGE values which are biomarkers of MOA, e.g., AA Amyloidosis or AL amyloidosis, and which can be used singly or in combination.

Interleukin-18 (IL-18)

IL-18 is a pleiotropic pro-inflammatory cytokine that activates B-cells to produce IFN-gamma and inhibits IgE production. IL-18 has been shown to strongly augment the production of IFN-gamma by T-cells and NK cells.

Interleukin-18 (IL-18) has been implicated in the T helper I response, in subjects with rheumatoid arthritis (RA), with or without amyloidosis. Plasma IL-18 levels were studied by enzyme-linked immunosorbent assay in RA subjects with reactive amyloidosis and in RA subjects without amyloidosis matched with respect to age, sex, seropositivity, disease duration and inflammatory activity, as well as in healthy control subjects it was shown that plasma IL-18 levels were significantly elevated in RA subjects as compared with healthy control subjects. Those RA subjects who had amyloid had a significantly higher circulating level of IL-18 than those without amyloid (418.1+/−32.1 ng/l versus 317.0+/−21.3 ng/l, P<0.02) and the difference in IL-18 was not due to differences in inflammatory activity, nor was it related to renal function (Maury, C. P. (2002) Amyloid. 9:141).

Accordingly, suitable biomarkers of MOA, e.g., AA amyloidosis that can be employed in accordance with the methods and kits of the invention include an elevated level of serum IL-18.

Macrophage-Colony Stimulating Factor (M-CSF)

Macrophage-Colony Stimulating Factor (M-CSF) is a colony-stimulating factor, secreted by macrophages, stimulated endothelial cells, and most tissues. It stimulates the production of macrophages from precursor cells and maintains the viability of mature macrophages in vitro. The abundance and activation of macrophages in the inflamed synovial membrane/pannus significantly correlates with the severity of rheumatoid arthritis (RA). Although unlikely to be the ‘initiators’ of RA, macrophages possess widespread pro-inflammatory, destructive, and remodeling capabilities that can critically contribute to acute and chronic disease. Also, activation of the monocytic lineage is not locally restricted, but extends to systemic parts of the mononuclear phagocyte system.

Sera and urine samples from subjects with secondary amyloidosis (AA), primary amyloidosis (AL), systemic autoimmune diseases with renal impairment and healthy controls were analyzed. Plasma levels of M-CSF in the AA group were significantly increased in comparison to healthy controls (1077.34+/−238.6 vs. 137.71+/−19.6, pg/mL, p<0.001) and also in comparison to the group with systemic autoimmune diseases (482.24+/−86.7 pg/mL, p<0.05). Urinary excretions of M-CSF (650.2+/−153.7 vs. 33.3+/−8.6 micrograms/mol creatinine, p<0.01) in AA were significantly increased in comparison to controls. Subjects with AL had increased plasma levels of M-CSF (819.83+/−264.2 vs. 137.71+/−19.6 pg/mL, p<0.05) and increased urinary excretion of M-CSF (865.0+/−188.4 vs. 33.3+/−8.6 micrograms/mol creatinine, p<0.01) in comparison to controls (Rysava, R., et al. (1999) Biochem Mol Biol Int. 47:845).

Accordingly, suitable biomarkers of MOA, e.g., AA amyloidosis that can be employed in accordance with the methods and kits of the invention include an elevated level of plasma M-CSF.

Antibody Against Citrulinated Vimentin (Sa)

Antibodies directed to the Sa antigen, or citrullinated vimentin, are highly specific for rheumatoid arthritis (RA) and can be detected in approximately 40% of RA sera. These antibodies appear to identify a subset of early rheumatoid arthritis subjects destined to have aggressive and destructive disease.

The Sa antigen, a doublet of protein bands of about 50 kDa, is present in placenta and in RA synovial tissue (Vossenaar, E. R., et al., Arthritis Res Ther. (2004) 6:R142-50). Anti-Sa antibodies are detected in the serum of 20-47% of subjects with rheumatoid arthritis. It has recently been confirmed that anti-Sa antibodies are directed to citrullinated vimentin, thus placing them in the anti-citrulline family of autoantibodies. The Sa antigen has previously been shown to be present in synovium. This, along with the demonstration of citrullinated proteins in rheumatoid synovium, suggests that anti-Sa antibodies may play a pathogenic role in the initiation and/or persistence of rheumatoid synovitis (El-Gabalawy H S, Wilkins J A. Arthritis Res Ther (2004) 6:86).

Accordingly, suitable biomarkers of MOA, e.g., AA amyloidosis that can be employed in accordance with the methods and kits of the invention include the presence of autoantibody against citrulinated vimentin.

Monoclonal Immunoglobulin L Chain

In approximately 50% of patients monoclonal immunoglobulin L chain is detected on routine clinical laboratory testing of the serum or the urine. The most common precursor proteins are L chains of the lambda (λ) class. Lambda AL is approximately twice as prevalent as kappa (κ) AL, and L chains of the V_{λ}6 class are the most amyloidogenic. Clonal plasma cell proliferative diseases in which the V_{λ}66 gene is expressed are always associated with amyloid deposition. Among V_{κ} genes, the V_{κ}1 subgroup is overrepresented among amyloid-forming L chains.

Within the V region families, certain amino acid residues occurring at particular positions in the L-chain sequence render those chains more amyloidogenic, with a combination of such residues increasing the chances of a particular L-chain protein being associated with tissue amyloid deposition. Another structural feature that appears to predispose to AL deposition is enzymatic glycosylation of the L chain. While 15% of human L chains bear sugar residues, almost one third of amyloidogenic L chains are glycosylated. Why certain amino acid and glycosylation characteristics in L chains predispose to amyloid formation remains unknown.

AL deposits contain intact L chains, L-chain fragments, or both (most patients). The fragments always include the amino terminus of the chain and range in mass from 5000-16,000 Da. In 90% of patients, the deposited peptides include at least some constant region sequence; therefore, the peptides react with commercially available anti-L chain sera, which are specific for constant region determinants. When an intact whole monoclonal immunoglobulin is present in serum, the concentration is less than 10 g/l in 30% of patients, less than 20 g/l in over 70% of patients and above 30 g/l in less than 10% (Kyle & Gertz, (1995) Seminars in Hematology 32:45-59).

Accordingly, suitable biomarkers of MOA, e.g., AL amyloidosis that can be employed in accordance with the methods and kits of the invention include the presence of monoclonal immunoglobulin L chain in the serum and/or urine.

Albuminuria and Creatinine Clearance

As discussed above, renal dysfunction is one of the most common presenting features of patients with MOA. While not wishing to be bound by theory, deposition of amyloid fibrils, e.g., AA or AL fibrils, in the kidney damages the basement membrane and/or components of the glomerular epithelial system, and often the first manifestation of this is the appearance of plasma proteins in the urine. Because albumin is the major circulating protein in plasma and is relatively close in size to that of the size selectivity barrier in the kidney, its appearance in the urine is the most sensitive indicator of damage or disruption of the glomerular filtration barrier. The normal urinary excretion of albumin is less than 30 mg/dl, and urinary excretion of albumin greater than or equal to 300 mg/dl (albuminuria) is an indication of kidney malfunction and/or damage, and can therefore be used according to the methods of the present invention as a biomarker of MOA, e.g., AA amyloidosis or AL amyloidosis.

Furthermore, reduction in renal blood flow that controls glomerular pressure and hence glomerular filtration will also be affected by the deposition of amyloid fibrils. Since changes in serum creatinine reflect changes in glomerular filtration rate, measurement of creatinine clearance is an effective measure of kidney function.

Normal creatinine clearance, measured as the amount of creatinine in serum, is 1.0 mg/dl. Serum creatinine that is 2.0 mg/dl represents approximately a 50% reduction in glomerular filtration rate.

Accordingly, suitable biomarkers of MOA, e.g., AA amyloidosis or AL amyloidosis, that can be employed in accordance with the methods and kits of the invention include increased urinary albumin, e.g., albuminuria, and increased creatinine clearance, e.g., serum creatinine, which are biomarkers of MOA, e.g., AA amyloidosis or AL amyloidosis, and which can be used singly or in combination.

### METHODS OF THE INVENTION

The present invention provides methods of assessing whether a subject has or is at risk of having MOA, e.g., AL amyloidosis or AA Amyloidosis. In one embodiment, this method includes assaying a plurality of biomarkers of MOA, e.g., AA amyloidosis or AL Amyloidosis, wherein the detection of at least two of the biomarkers indicates the subject has or is at risk of having MOA, e.g., AA amyloidosis or AL Amyloidosis. “Detection”, as used herein, refers to assaying for a modulation, e.g., upmodulation and downmodulation, of a biomarker of the invention relative to an appropriate control. Detection is also meant to include instances when a biomarker of the invention becomes present and/or absent when compared to an appropriate control.

The biomarkers of the invention generally are DNA, RNA, protein and autoantibody molecules which can be detected in one or both of normal and MOA, e.g., AA amyloidosis or AL amyloidosis, subjects. The presence, absence, or level of at least two of these biomarkers in a sample, e.g., a sample containing, e.g., blood, e.g., plasma and/or serum, urine, stool, cerebrospinal (i.e. CSF) and spinal fluid, synovial fluid, conjunctival fluid, salivary fluid, lymph, bile, tears, and sweat, tissues or cells, e.g., monocytes and epithelial cells, e.g., gingival epithelial cells, is herein correlated with the state of the subject. The invention thus provides compositions, kits, and methods for assessing the disease state of subjects.

Accordingly, the invention provides compositions, kits, and methods for at least the following:

1) assessing whether a subject is at risk of developing or has developed MOA, e.g., AA amyloidosis or AL amyloidosis;

2) assessing the relative efficacy of one or more therapeutic agents or drugs in inhibiting MOA, e.g., AA amyloidosis or AL amyloidosis, or deposition of amyloid, e.g., AA or AL amyloid, in a subject;

3) assessing the efficacy of a therapy for modulating or inhibiting MOA, e.g., AA amyloidosis or AL amyloidosis, or deposition of amyloid, e.g., AA or AL amyloid in a subject;

4) monitoring the progression, stabilization or regression of MOA, e.g., AA amyloidosis or AL amyloidosis in a subject; and/or

5) selecting a composition or therapy for modulating or inhibiting MOA, e.g., AA amyloidosis or AL amyloidosis, or deposition of amyloid, e.g., AA or AL amyloid in a subject.

The invention thus includes a method of assessing whether a subject is at risk of developing MOA, e.g., AA amyloidosis or AL amyloidosis. In one embodiment, this method comprises comparing the presence or absence of a biomarker variant allele and/or biomarker autoantibody and/or level of a biomarker nucleic acid and/or protein in a subject sample and the presence or absence of a biomarker variant allele and/or biomarker autoantibody and/or level of the biomarker nucleic acid and/or protein in a suitable control, e.g., one or several non-MOA amyloidosis samples or a benchmark based on the same. The presence of a biomarker variant allele and/or biomarker autoantibody and/or the absence of a biomarker variant allele and/or biomarker autoantibody and/or a significant difference between the level of the biomarker nucleic acid and/or protein in the subject sample and the normal level of the biomarker nucleic acid and/or protein is an indication that the subject is afflicted with and/or at risk of developing MOA, e.g., AA amyloidosis or AL amyloidosis. The AA amyloidosis biomarker is selected from the group consisting of the presence of a variant SAA allele; an elevated serum amyloid A protein (SAA) level; a depressed glycosaminoglycan (GAG) level in urine; an elevated interleukin-18 (IL-18) level; an elevated macrophage-colony stimulating factor (M-CSF) level; an elevated hepatocyte growth factor (HGF) level; and the presence of an autoantibody against citrulinated vimentin (Sa), wherein the detection of at least two of these biomarkers indicates a diagnosis of AA amyloidosis. The AL amyloidosis biomarker is selected from the group consisting of a depressed glycosaminoglycan (GAG) level in urine; an elevated hepatocyte growth factor (HGF) level; an increase in serum albumin, e.g., albuminuria, an increase in creatinine clearance, and the presence of a monoclonal immunoglobulin light chain in the serum or urine, wherein the detection of at least two of these biomarkers indicates a diagnosis of AL amyloidosis. Although one or more molecules corresponding to the biomarkers of the invention (described above) may have been described by others, none of these biomarkers in isolation is as specific and/or as sensitive, as is the combination of, e.g., two or more, of these biomarkers with regard to the presence of MOA, e.g., AA amyloidosis or AL amyloidosis, in a subject and has not previously been recognized.

Any biomarker or combination of biomarkers may be used in the compositions, kits, and methods of the present invention. In general, it is preferable to use biomarkers for which the difference between the level of the biomarker in a biomarker-associated disease subjects, e.g., MOA subjects, e.g., AA amyloidosis subjects or AL amyloidosis subjects, and the level of the same biomarker in normal subjects is as great as possible. Although this difference can be as small as the limit of detection of the method for assessing expression of the biomarker, it is preferred that the difference be at least greater than the standard error of the assessment method, and preferably a difference of at least 2-, 3-, 4-, 5-, 6-, 7-, 8-, 9-, 10-, 15-, 20-, 25-, 50-, 100-, 500-, 1000-fold or greater than the level of the same biomarker in normal subjects.

Techniques for detection of a biomarker protein include introducing into a subject or a biopsy or other sample a labeled antibody directed against the protein. For example, the antibody can be labeled with a radioactive biomarker whose presence and location in a subject can be detected by standard imaging techniques, such as, for example, PET and scintigraphy.

Certain biomarkers identified by the methods of the invention may be secreted proteins. It is a simple matter for the skilled artisan to determine whether any particular biomarker protein is a secreted protein. In order to make this determination, the biomarker protein is expressed in, for example, a mammalian cell, preferably a human cell line, extracellular fluid is collected, and the presence or absence of the protein in the extracellular fluid is assessed (e.g. using a labeled antibody which binds specifically with the protein).

It will be appreciated that subject samples, e.g., a sample containing, e.g., blood, e.g., plasma and/or serum, urine, stool, cerebrospinal (i.e. CSF) and spinal fluid, synovial fluid, conjunctival fluid, salivary fluid, lymph, bile, tears, and sweat, may contain cells, e.g. monocytes and epithelial cells, therein, and thus may be used in the methods of the present invention. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the presence and/or level of a biomarker in the sample.

In one embodiment, the invention includes compositions, kits, and/or methods employed to assay or to detect the presence and/or level of biomarkers corresponding to proteins having at least one portion which is displayed on the surface of cells which expresses it. It is a simple matter for the skilled artisan to determine whether the protein corresponding to any particular biomarker comprises a cell-surface protein. For example, immunological methods may be used to detect such proteins on whole cells, or well-known computer-based sequence analysis methods (e.g. the SIGNALP program; Nielsen et al., 1997, Protein Engineering 10:1-6) may be used to predict the presence of at least one extracellular domain (i.e. including both secreted proteins and proteins having at least one cell-surface domain). Expression, or the presence, of a biomarker corresponding to a protein having at least one portion which is displayed on the surface of a cell which expresses it may be detected without necessarily lysing the cell (e.g. using a labeled antibody which binds specifically with a cell-surface domain of the protein).

The presence, absence, and/or level of a biomarker of the invention may be assessed by any of a wide variety of well known methods for detecting a transcribed molecule, protein or autoantibody. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods, ELISA, immunoblotting, Western blotting, Northern blotting, Southern blotting and the like.

In one embodiment, the presence, absence, and/or level of a biomarker is assessed using an antibody (e.g. a radio-labeled, chromophore-labeled, fluorophore-labeled, or enzyme-labeled antibody), an antibody derivative (e.g. an antibody conjugated with a substrate or with the protein or ligand of a protein-ligand pair (e.g. biotin-streptavidin)), or an antibody fragment (e.g. a single-chain antibody, an isolated antibody hypervariable domain, etc.) which binds specifically with a protein corresponding to the biomarker, such as the protein encoded by the open reading frame corresponding to the biomarker or such a protein which has undergone all or a portion of its normal post-translational modification.

In another embodiment, expression of a biomarker is assessed by preparing genomic DNA or mRNA/cDNA (i.e. a transcribed polynucleotide) from cells in a subject sample, and by hybridizing the genomic DNA or mRNA/cDNA with a reference polynucleotide which is a complement of a polynucleotide comprising the biomarker, and fragments thereof. cDNA can, optionally, be amplified using any of a variety of polymerase chain reaction methods prior to hybridization with the reference polynucleotide. Expression of one or more biomarkers can likewise be detected using quantitative PCR (e.g., QPCR) to assess the level of expression of the biomarker(s). Alternatively, any of the many known methods of detecting mutations or variants (e.g. single nucleotide polymorphisms, deletions, etc.) of a biomarker of the invention may be used to detect occurrence of a biomarker in a subject.

The present invention also relates to the detection of the presence and/or absence of allelic variants. In one embodiment, the methods of the invention can be used to identify the presence and/or absence of a specific allelic variant of one or more polymorphic regions of a gene in a biological sample. The allelic differences can be: (i) a difference in the identity of at least one nucleotide or (ii) a difference in the number of nucleotides, which difference can be a single nucleotide or several nucleotides. The invention also provides methods for detecting differences in a gene such as chromosomal rearrangements, e.g., chromosomal dislocation.

A preferred detection method is allele specific hybridization using probes overlapping the polymorphic site and having about 5, 10, 20, 25, or 30 nucleotides around the polymorphic region. In a preferred embodiment of the invention, several probes capable of hybridizing specifically to allelic variants are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al., (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment. For example, the identity of the allelic variant of a nucleotide polymorphism in a 5′ upstream regulatory element can be determined in a single hybridization experiment.

In certain aspects of the methods of the invention, it is necessary to first amplify at least a portion of a gene prior to detecting an allelic variant. Amplification can be performed, e.g., by PCR and/or LCR (see Wu and Wallace, (1989) Genomics 4:560), according to methods known in the art. In one embodiment, DNA and/or RNA of a cell is exposed to two PCR primers and amplified for a number of cycles sufficient to produce the required amount of amplified DNA and/or cDNA. In some embodiments, the primers are located between 100 and 350 base pairs apart.

In some cases, the presence and/or absence of an allelic variant of a gene in DNA from a subject can be shown by restriction enzyme analysis. For example, a specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide with piperidine) can be used to detect mismatched bases in RNA/RNA DNA/DNA, or RNA/DNA heteroduplexes (Myers, et al., (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of an allelic variant with a sample nucleic acid, e.g., RNA or DNA, obtained from a tissue sample. The double-stranded duplexes are treated with an agent that cleaves single-stranded regions of the duplex such as regions formed based on base pair mismatches between the control and sample strands. See, for example, Cotton et al., (1988) Proc. Natl. Acad. Sci., USA 85:4397; Saleeba, et al., (1992) Methods Enzymol. 217:286-295. In one embodiment, the control or sample nucleic acid is labeled for detection.

In another embodiment, an allelic variant can be detected by denaturing high-performance liquid chromatography (DHPLC) (Oefner and Underhill, (1995) Am. J. Human Gen. 57:Suppl. A266). DHPLC uses reverse-phase ion-pairing chromatography to detect the heteroduplexes that are generated during amplification of PCR fragments from individuals who are heterozygous at a particular nucleotide locus within that fragment (Oefner and Underhill (1995) Am. J. Human Gen. 57:Suppl. A266). In general, PCR products are produced using PCR primers flanking the DNA of interest. DHPLC analysis is carried out and the resulting chromatograms are analyzed to identify base pair alterations or deletions based on specific chromatographic profiles (see O'Donovan, et al., (1998) Genomics 52:44-49).

In other embodiments, alterations in electrophoretic mobility are used to detect the type of allelic variant. For example, single strand conformation polymorphism (SSCP) may be used to detect differences in electrophoretic mobility between mutant and wild-type nucleic acids (Orita et al., (1989) Proc. Natl. Acad. Sci., USA 86:2766; see also Cotton (1993) Mutat Res. 285:125-144; and Hayashi (1992) Genet. Anal. Tech. Appl. 9:73-79). In another preferred embodiment, the subject method utilizes heteroduplex analysis to separate double stranded heteroduplex molecules on the basis of changes in electrophoretic mobility (Keen, et al., (1991) Trends Genet. 7:5).

Other examples of techniques for identifying and/or detecting differences of at least one nucleotide between two nucleic acids include, but are not limited to, selective oligonucleotide hybridization, selective amplification, or selective primer extension.

In another embodiment, a combination of methods to assess the presence, absence or level of a biomarker is utilized.

Because the compositions, kits, and methods of the invention rely on detection of a difference in levels of one or more biomarkers of the invention, it is preferable that the level of the biomarker is significantly greater than the minimum detection limit of the method used to assess expression in at least one of normal subject sample and an MOA subject, e.g., AA amyloidosis subject or AL amyloidosis subject, sample.

In addition, as a greater number of subject samples are assessed for expression of the biomarkers of the invention and the outcomes of the individual subjects from whom the samples were obtained are correlated, it will also be confirmed that altered levels of certain of the biomarkers of the invention are strongly correlated with MOA, e.g., AA amyloidosis or AL amyloidosis, and that altered level of other biomarkers of the invention are not strongly correlated with MOA, e.g., AA amyloidosis or AL amyloidosis.

It is recognized that the compositions, kits, and methods of the invention will be of particular utility to subjects having an enhanced risk of developing MOA, e.g., AA amyloidosis or AL amyloidosis, and their medical advisors. Subjects recognized as having an enhanced risk of developing MOA, e.g., AA amyloidosis or AL amyloidosis, include, for example, subjects having a familial history of AA amyloidosis, subjects with chronic inflammatory disease, and subjects that do not respond to disease-modifying antirheumatic drugs (DMARDs), subjects with chronic inflammatory disease, subjects with familial or hereditary form of amyloidosis, e.g., FMF (AA amyloidosis), as well as subjects with multiple myeloma or a plasma cell dyscrasia (AL amyloidosis)

As further information becomes available as a result of routine performance of the methods described herein, population-average values for normal levels of the biomarkers of the invention may be used. In other embodiments, the ‘normal’ level of a biomarker may be determined by assessing the level of the biomarker in a subject sample obtained from a non-MOA, e.g., non-AA amyloidosis- or non-AL amyloidosis-afflicted subject, from a subject sample obtained from a subject before the suspected onset of MOA, e.g., AA amyloidosis or AL amyloidosis, in the subject from archived subject samples, and the like.

The invention includes compositions, kits, and methods for assessing the presence of MOA, e.g., AA amyloidosis or AL amyloidosis, in a subject sample (e.g. an archived tissue sample or a sample obtained from a subject). These compositions, kits, and methods are substantially the same as those described above, except that, where necessary, the compositions, kits, and methods are adapted for use with samples other than subject samples. For example, when the sample to be used is a parafinized, archived human tissue sample, it can be necessary to adjust the ratio of compounds in the compositions of the invention, in the kits of the invention, or the methods used to assess levels of biomarker expression in the sample. Such methods are well known in the art and within the skill of the ordinary artisan.

The invention includes a kit for assessing the presence of MOA, e.g., AA amyloidosis or AL amyloidosis in a subject (e.g. in a sample such as a subject sample). The kit comprises a plurality of reagents, each of which is capable of binding specifically with a nucleic acid or polypeptide corresponding to a biomarker of the invention. Suitable reagents for binding with a polypeptide corresponding to a biomarker of the invention include, e.g., antibodies, antibody derivatives, antibody fragments, and the like. Suitable reagents for binding with a nucleic acid (e.g. a genomic DNA, an mRNA, a spliced mRNA, a cDNA, or the like) include, e.g., complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like.

The kit of the invention may optionally comprise additional components useful for performing the methods of the invention. By way of example, the kit may comprise fluids (e.g. SSC buffer) suitable for annealing complementary nucleic acids or for binding an antibody with a protein with which it specifically binds, one or more sample compartments, an instructional material which describes performance of a method of the invention, a normal subject sample, e.g., a sample from a subject that does not have and/or is not at risk of developing MOA, e.g., AA amyloidosis or AL amyloidosis, a sample from a subject in which MOA, e.g., AA amyloidosis or AL amyloidosis, has been confirmed by, for example, biopsy, and the like.

The invention also includes a method of assessing the therapy for modulating or inhibiting MOA, e.g., AA amyloidosis or AL amyloidosis, in a subject. Compounds or treatments, e.g., chemotherapy, which modulate or inhibit MOA, e.g., AA amyloidosis or AL amyloidosis, in a subject may cause the level of the biomarkers of the invention to change to a level nearer or further from a suitable control, e.g., a normal level for that biomarker, i.e. the level for the biomarker in non-MOA subjects, e.g., non-AA amyloidosis or non-AL amyloidosis subjects. In one embodiment of this method, the level of at least two of the biomarkers of the invention in a pair of samples (one subjected to the therapy, the other not subjected to the therapy) is assessed. If the therapy induces a significant depression or elevation in the level of one or more biomarkers of the invention, this is an indication that the therapy is efficacious for modulating MOA, e.g., AA amyloidosis or AL amyloidosis. This method may be employed to assess alternative and/or additional therapies in patient samples (e.g., patient blood and/or tissue) in order to select a therapy most likely to be efficacious for inhibiting MOA, e.g., AA amyloidosis or AL amyloidosis, in a particular subject or may be employed to generally assess different therapies, e.g., different therapeutic compounds, for treating MOA, e.g., AA amyloidosis or AL amyloidosis.

In another embodiment of the present invention, the methods described herein, may be utilized for screening test compounds, i.e., candidate or test compounds or agents (e.g., peptides, peptidomimetics, small molecules (organic or inorganic) or other drugs) useful for modulating MOA, e.g., AA amyloidosis or AL amyloidosis. The method may comprise contacting a cell with a candidate compound and the level and/or presence and/or absence of a biomarker of the invention in the cell is determined. The level and/or presence and/or absence of the biomarker of the invention in the presence of the candidate compound is compared to the level and/or presence and/or absence of a biomarker of the invention in the absence of the candidate compound. The candidate compound can then be identified as a modulator of MOA, e.g., AA amyloidosis or AL amyloidosis, based on this comparison.

Confirmation and Monitoring of MOA

Subjects may present with a profile of high levels in two or more of these biomarkers. These levels would be different than that seen in healthy individuals and in subjects with chronic inflammatory or infectious disease without MOA, e.g., AA amyloidosis, and in subjects with plasma cell dyscrasia or multiple myeloma without MOA, e.g., AL amyloidosis. Monitoring of the level of GAGs in urine can also serve a second purpose. GAGs mimetic compounds have been shown to be anti-amyloid agents. Measurement of GAGs in urine could thus be used to monitor subjects treated with GAGs mimetics (e.g., propane-1,3-disulfonic acid and disodium propane-1,3-disulfonate). Responders to this anti-amyloid therapy should show increased GAGs levels in urine since the compound would block association of GAGs to the amyloid protein. Monitoring urine GAGs could serve two purposes: identifying subjects who respond well to therapy, and evaluating regression of amyloidosis in treated subjects during therapy.

In one embodiment, after the present method has been employed to indicate the diagnosis of MOA, e.g., AA amyloidosis or AL amyloidosis, the methods of the invention optionally include the step of confirming the diagnosis of MOA, e.g., AA amyloidosis or AL amyloidosis by biopsy. By employing the methods and/or kits of the present invention, a biopsy need only be performed after the safe and non-invasive methods of the present invention have indicated diagnosis.

Conventional biopsy procedures include rectal biopsy, subcutaneous fat biopsy, labial salivary gland biopsy, small bowel biopsy, stomach biopsy, renal biopsy, liver biopsy, and endomyocardial biopsy. Biopsy tissue samples may be stained to yield structural properties (e.g., by staining with haematoxylin), birefringence (e.g., by staining with Congo red viewed under polarized light), or characteristic metachromasia (e.g., by staining with thioflavine-T or thioflavine-S).

Additionally or alternatively, the presence and extent of amyloid deposition generally can be detected by injecting the subject with radio-iodinated purified SAP, and scanning with a gamma camera. The presence and extent of deposition can then be detected as SAP binds to amyloid fibrils in vivo. Cunnane & Whitehead, Bailliere's Clinical Rheumatology 13(4) 615-28 (1999).

In one embodiment, after diagnosis of MOA, e.g., AA amyloidosis or AL amyloidosis, employing the methods of the present invention, the invention can further include monitoring the progression of MOA, e.g., AA amyloidosis or AL amyloidosis, or response to treatment of MOA, e.g., AA amyloidosis or AL amyloidosis, using a gamma scan.

Predictive Medicine

The present invention pertains to the field of predictive medicine in which diagnostic assays, prognostic assays, and monitoring clinical trails are used for prognostic (predictive) purposes to thereby treat an individual prophylactically. Accordingly, one aspect of the present invention relates to diagnostic assays for determining the level of expression of polypeptides or nucleic acids corresponding to one or more biomarkers of the invention, in order to determine whether an individual is at risk of developing MOA, e.g., AA amyloidosis or AL amyloidosis. Such assays can be used for prognostic or predictive purposes to thereby prophylactically treat an individual prior to the onset of MOA, e.g., AA amyloidosis or AL amyloidosis.

Yet another aspect of the invention pertains to monitoring the influence of therapy, e.g., chemotherapy and/or agents, e.g., drugs or other compounds, administered either to inhibit MOA, e.g., AA amyloidosis or AL amyloidosis, or to treat or prevent any other disorder on the level of a biomarker of the invention in clinical trials.

Diagnostic Assays

An exemplary method for detecting the presence or absence of a biomarker variant allele and/or biomarker autoantibody or the level of a polypeptide or nucleic acid corresponding to a biomarker of the invention in a biological sample involves obtaining a biological sample, e.g., a sample containing e.g., blood, e.g., plasma and/or serum, urine, stool, cerebrospinal (i.e. CSF) and spinal fluid, synovial fluid, conjunctival fluid, salivary fluid, lymph, bile, tears, and sweat, tissues or cells, e.g., monocytes and epithelial cells, e.g., gingival epithelial cells, from a test subject and contacting the biological sample with a compound or an agent capable of detecting the biomarker variant allele and/or biomarker autoantibody or the level of a polypeptide or nucleic acid (e.g., mRNA, genomic DNA, or cDNA). The detection methods of the invention can thus be used to detect mRNA, protein, antibody, cDNA, or genomic DNA, for example, in a biological sample in vitro as well as in vivo. For example, in vitro techniques for detection of mRNA include Northern hybridizations, in situ hybridizations and QPCR. In vitro techniques for detection of a polypeptide or antibody corresponding to a biomarker of the invention include, for example, enzyme linked immunosorbent assays (ELISAs), Western blots, immunoprecipitations and immunofluorescence. In vitro techniques for detection of genomic DNA include, for example, Southern hybridizations. Furthermore, in vivo techniques for detection of a polypeptide or antibody corresponding to a biomarker of the invention include introducing into a subject a labeled antibody directed against the polypeptide or antibody. For example, the antibody can be labeled with a radioactive biomarker whose presence and location in a subject can be detected by standard imaging techniques.

A general principle of such diagnostic and prognostic assays involves preparing a sample or reaction mixture that may contain a biomarker, and a probe, under appropriate conditions and for a time sufficient to allow the biomarker and probe to interact and bind, thus forming a complex that can be removed and/or detected in the reaction mixture. These assays can be conducted in a variety of ways.

For example, one method to conduct such an assay would involve anchoring the biomarker or probe onto a solid phase support, also referred to as a substrate, and detecting target biomarker/probe complexes anchored on the solid phase at the end of the reaction. In one embodiment of such a method, a sample from a subject, which is to be assayed for presence and/or concentration of biomarker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, biomarker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the biomarker or probe belongs. Well-known supports or carriers include, but are not limited to, glass, polystyrene, nylon, polypropylene, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of biomarker/probe complexes anchored to the solid phase can be accomplished in a number of methods outlined herein.

In a preferred embodiment, the probe, when it is the unanchored assay component, can be labeled for the purpose of detection and readout of the assay, either directly or indirectly, with detectable labels discussed herein and which are well-known to one skilled in the art.

It is also possible to directly detect biomarker/probe complex formation without further manipulation or labeling of either component (biomarker or probe), for example by utilizing the technique of fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a biomarker can be accomplished without labeling either assay component (probe or biomarker) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with biomarker and probe as solutes in a liquid phase. In such an assay, the complexed biomarker and probe are separated from uncomplexed components by any of a number of standard techniques, including but not limited to: differential centrifugation, chromatography, electrophoresis and immunoprecipitation. In differential centrifugation, biomarker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the biomarker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl 1997 Oct. 10; 699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the presence or level of a biomarker of the invention can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject.

In another embodiment of the present invention, a polypeptide corresponding to a biomarker is detected. A preferred agent for detecting a polypeptide of the invention is an antibody capable of binding to a polypeptide corresponding to a biomarker of the invention, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Proteins can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining whether cells express a biomarker of the present invention.

In one format, antibodies, or antibody fragments, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from, e.g., epithelial cells, can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

The invention also encompasses kits for detecting the presence of a biomarker variant allele and/or autoantibody and/or a polypeptide or nucleic acid corresponding to a biomarker of the invention in a biological sample, e.g., a sample containing e.g., blood, e.g., plasma and/or serum, urine, stool, cerebrospinal (i.e. CSF) and spinal fluid, synovial fluid, conjunctival fluid, salivary fluid, lymph, bile, tears, and sweat, tissues or cells, e.g., monocytes and epithelial cells, e.g., gingival epithelial cells. Such kits can be used to determine if a subject is suffering from or is at increased risk of developing MOA, e.g., AA amyloidosis or AL amyloidosis. For example, the kit can comprise a labeled compound or agent capable of detecting a polypeptide or an mRNA encoding a polypeptide corresponding to a biomarker of the invention in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

For antibody-based kits, the kit can comprise, for example: (1) a first antibody (e.g., attached to a solid support) which binds to a polypeptide corresponding to a biomarker of the invention; and, optionally, (2) a second, different antibody which binds to either the polypeptide or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a polypeptide corresponding to a biomarker of the invention or (2) a pair of primers useful for amplifying a nucleic acid molecule corresponding to a biomarker of the invention. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

Pharmacogenomics

Agents or modulators which have a stimulatory or inhibitory effect on expression of a biomarker of the invention can be administered to individuals to treat (prophylactically or therapeutically) MOA, e.g., AA amyloidosis or AL amyloidosis in the subject. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the level of expression of a biomarker of the invention in an individual can be determined to thereby select appropriate agent(s) for therapeutic or prophylactic treatment of the individual.

Pharmacogenomics deals with clinically significant variations in the response to drugs due to altered drug disposition and abnormal action in affected persons. See, e.g., Linder (1997) Clin. Chem. 43(2):254-266. In general, two types of pharmaco-genetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some subjects do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the level of a biomarker of the invention in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of expression of a biomarker of the invention.

Monitoring Clinical Trials

Monitoring the influence of agents (e.g., drug compounds) on the level of expression of a biomarker of the invention can be applied not only in basic drug screening, but also in clinical trials. This method may also be employed to otherwise monitor the treatment of any subject. In one embodiment, the effectiveness of an agent to affect the level of a biomarker of the invention can be monitored in clinical trials of subjects receiving treatment for MOA, e.g., AA amyloidosis or AL Amyloidosis. In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate). Exemplary methods may include one or more of the following steps: (i) obtaining a pre-administration sample from a subject prior to administration of the agent; (ii) detecting the level of two or more of the biomarkers of the invention in the pre-administration sample; (iii) obtaining one or more post-administration samples from the subject; (iv) detecting the level of two or more of the biomarkers of the invention in the post-administration samples; (v) comparing the level of the biomarker in the pre-administration sample with the level of the marker(s) in the post-administration sample or samples; and (vi) altering the administration of the agent to the subject accordingly. For example, increased administration of the agent can be desirable to increase the level of the biomarker to higher levels than detected, i.e., to increase the effectiveness of the agent. Alternatively, decreased administration of the agent can be desirable to decrease expression of the biomarker(s) to lower levels than detected, i.e., to decrease the effectiveness of the agent.

This invention is further illustrated by the following examples which should not be construed as limiting. The contents of all references, patents and published patent applications cited throughout this application are incorporated herein by reference.

### EXAMPLES

### Example 1

### Biomarkers Present in a Sample of Multiple Organ Amyloidosis Subjects

Biomarkers Present in the Urine and/or Serum of AA Amyloidosis Subjects

This example describes a non-investigational treatment study. Patients diagnosed with AA amyloidosis will also be part of the trial. Profile of markers obtained in patients with AA amyloidosis will be compared to those obtained in the present subset of patients with RA. The subset of interest for this portion of the study is only RA patients.

Patients diagnosed with rheumatoid arthritis (RA) will be recruited from 6-10 sites in the province of Québec. Samples of blood and urine will be collected from them at two time points, i.e. at baseline and at 3 to 6 months after the first sample collection. The first group of RA patients is at the lowest risk of having developed AA amyloidosis (LRRA) and the second group of RA patients represents a cohort which includes individuals at the highest risk of having developed AA amyloidosis (HRRA).

1. AA Group: Patients with a diagnosis of AA amyloidosis confirmed by positive Congo red staining and immunohistochemistry or immunoelectron microscopy of biopsied material.

2. LRRA Group: Patients with a diagnosis of rheumatoid arthritis (RA) for up to 8 years. As a result of their recent diagnosis of RA, these patients are at low risk of AA amyloidosis.

3. HRRA Group: Patients with a diagnosis of RA of 15 years or more and having active disease as defined by either:
Patients failed 3 or more Disease Modifying Anti-Rheumatic Drugs (DMARDs) and/or;
Patients have a HAQ score of 1.5 or more.
   This group of patients is at a higher risk of AA amyloidosis as their diagnosis of RA is long standing and their disease is not well controlled as defined by the ongoing activity of their inflammatory disease.

Information on patients will also be collected in regards to demographics, medical history, current medical condition, past and concomitant medication, and several lab test results done within the past year.

Patients will need to give their written consent before any trial procedures are started. For the duration of this trial, patients will continue their regular treatments as prescribed and needed. General information of patients' demographics, current medical condition, disease history and concomitant medication will be recorded in a CRF (computer readable format). Once the samples are collected they will be sent to a third party for analysis.

The results (marker profile) of each RA group will be compared to one another and to those of patients diagnosed with AA amyloidosis.

Inclusion Criteria

To enter this study, patients must meet all of the following criteria:

1) Patients must be 18 years of age or older.

2) Patients must sign the written informed consent.

3) Women of childbearing potential will be eligible in this study.

However, should a patient become pregnant it will need to be reported as this condition may have an influence on the patient's blood and urine results.

In addition,

For the AA amyloidosis group:

Patients must have a diagnosis of AA amyloidosis demonstrated by positive biopsy (Congo red staining) and immunohistochemistry or immunoelectron microscopy. Tissue from previous biopsy can be used for confirmation of diagnosis.
   For the LRRA group:
Patients must have a diagnosis of RA for up to 8 years. The diagnosis of rheumatoid arthritis is based on the 1987 revised American College of Rheumatology (ACR) criteria. Patients will be considered as having RA if at least 4 of the following criteria are present at any time:
   i. Stiffness in and around the joints lasting 1 hour before maximal improvement, present for at least 6 weeks.
   ii. Arthritis of three or more joint areas, simultaneously, observed by a physician and present for at least 6 weeks.
   iii. Arthritis of the proximal interphalangeal (PIP), metacarpophalangeal (MCP), or wrist joints, present for at least 6 weeks.
   iv. Symmetric arthritis, present for at least 6 weeks.
   v. Rheumatoid nodules observed by a physician.
   vi. A positive test for serum rheumatoid factor (RF).
   vii. X-Ray changes characteristic of RA (erosion and/or periarticular osteopenia in hand and/or wrist joints).
      For the HRRA group:
Patients must have a diagnosis of RA (as defined above) of 15 years or more and at least one of the following criteria for disease activity:
   a. Failed 3 or more Disease Modifying Anti-Rheumatic Drugs (DMARDs).
   b. Has a Health Assessment Questionnaire (HAQ) score of 1.5 or more.
      Exclusion Criteria

1) Patients have reached end-stage renal disease or dialysis.

2) Patients have suspected or confirmed infection. Infection may affect the levels of some markers and therefore bias the results.

3) Patients suffer from active alcohol and/or drug abuse.

4) Patients have taken an investigational drug (including FibrillexTM) within the past, 3 months.

5) Patients have inability to provide legal consent.

Screening Visit

During the screening period, patient medical charts will be looked at to identify potential patients to participate in this trial. Inclusion and exclusion criteria will be the basis to select patients. Once a patient is identified and he/she is meeting one of the two RA groups described in the inclusion criteria, he/she will be explained the trial and will be offered to participate. If he/she agrees, patient will give written consent. Once this is done, study procedures may start.

Baseline Visit

Information on patient's demographics and history of disease with RA will be recorded on a CRF. Data such as time since patient's diagnosis and previous use of DMARDs will be recorded. Questions related to a patient's current medical condition such as morning stiffness and Health Assessment Questionnaire will be recorded on a CRF. All concomitant medication taken in the past 7 days will be recorded on a CRF.

Specifically:
Record patient demographics in the CRF:
   All patients: initials, year of birth, gender, body weight, height and ethnicity.
Record patient's RA or AA amyloidosis history in the CRF:
   RA patients: year of diagnosis, family history, number of joint(s) repaired.
   AA amyloidosis patients: year of diagnosis, description of underlying disease.
Record patient's current RA or AA amyloidosis disease condition in the CRF.
   RA patients: morning stiffness, rheumatoid factors and HAQ. Patients need to complete the HAQ with the assessment of pain and global disease activity.
   AA amyloidosis patients: clinical manifestations associated with AA amyloidosis.
   All patients: assessment of all systems for clinically relevant current disease or disorder.
Record lab test results in the CRF:
   Blood: albumin, hemoglobin, hematocrit, WBC, platelet count, serum creatinine, iron profile, protein electrophoresis, C-reactive protein (CRP).
   Urine: creatinine urea, microalbuminuria, proteinuria, calculated creatinine clearance (Cockcroft-Gault formula*).
      Cockcroft-Gault formula*:
      Creatinine Clearance(mL/min)=(140-age)×weight(kg)[×0.85 if women]
   serum creatinine (mg/dl)×72
Record past and concomitant medication:
   Past medication: All Disease Modifying Anti-Rheumatic Drugs (DMARDs) and anti-cytokine medications ever taken to treat RA or AA amyloidosis underlying disease.
   Current medication: all current medication taken at the time of recruitment (within the past month), including all over-the-counter medication (such as glucosamine).
Collect urine samples (refer to section 6.4).
Collect blood samples (refer to section 6.5).
Schedule patient for next visit in 2 to 4 months after baseline visit.
   Urine and Blood Tests Done in the Past Year

Results from the most recent urine and blood tests will be recorded on a CRF as long as these tests were done within the past year. The following parameters will be recorded: ALT, AST, ALP, total bilirubin, direct bilirubin, albumin, creatinine urea, proteinuria, (24-hour urine collection if available), CBC, PT, PTT, CRP, ESR, C3, C4, iron profile, protein electrophoresis, and the immunoglobulins (IgA, IgG, IgM).

Urine Sample Collection

For the analysis of the GAGs marker, sample of patient's urine will be collected. The urine containers to be used are Fisher Scientific—Cat #360 9874.

Blood Sample Collection

Blood samples will be drawn either by direct venipuncture or via an intravenous catheter.

Assessment of “Marker” Profiles

The following markers will be measured in each patient of the three groups:

Plasma levels:
Serum Amyloid A (SAA).
Hepatocyte Growth Factor (HGF).
Macrophage Colony Stimulating Factor (M-CSF).
Interleukin-18 (IL-18).
Autoantibodies against Citrullinated Vimentin (Sa).

Urine levels:
Glycosaminoglycans (GAGs).

The defined “marker” profile of the AA amyloidosis group will be compared to the one of the RA patient groups (LRRA and HRRA).

If as a result of the study, any RA patients are identified at high risk of having AA amyloidosis, the follow-up with those patients will be managed by the investigator.

Sample Size Calculation

Sample size calculations are generated for different markers of Diagnostic tests and were based on the following assumptions:

significance level of 5%;

power of 80%;

2-sided test procedure; and

minimum detectable difference of 20% and 30% between AA and HRRA groups.

For the markers which are quantitative in nature (SAA, M-CSF, HGF, IL-18 and GAG), sample sizes are generated from a Student-t Test and using standard deviations taken from the following references:
Rysava R et al., Biochem Mol Biol Int, 1999, 47(5), 845-850.
Shikano M et al., Intern Med 2000, 39(9), 715-719.
Tencer, Torffvit et al., Nephrol Dial Transplant, 1997, 12, 1161-1166.
Maury C P J et al., Amyloid: J. Protein Folding Disord. 2002, 9, 141-144.
Després et al., The Journal of Rheumatology, 1994, 21, 1027-1033.

For Sa (Vimentin), sample size is generated from a two sample Chi-square test (with continuity correction), assuming a proportion of 43% in the control group.

Minimum number of patients per group
to detect a difference of 20% and 30%

| MARKERS | 20% | 30% | 40% | 50% |
|---|---|---|---|---|
| SAA | 55 | 25 | N/A | N/A |
| M-CSF | 21 | 10 | N/A | N/A |
| HGF | 579 | 254 | 145 | 93 |
| GAG | 394 | 176 | N/A | N/A |
| IL-18 | 5 | 4 | N/A | N/A |
| Citrull. Vimentin | 107 | 48 | N/A | N/A |

For HGF, a sample size is calculated to detect a minimum difference of 40%. A sample size of n=165 patients per group is considered sufficient to conduct this study.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of assessing whether a subject has or is at risk of having multiple organ amyloidosis (MOA), comprising assaying a diagnostically predictive collection of biomarkers of multiple organ amyloidosis, wherein the detection of a diagnostically predictive collection of biomarkers of multiple organ amyloidosis indicates whether the subject has or is at risk of having multiple organ amyloidosis.

2.-57. (canceled)

58. The method of claim 1, comprising assaying at least two biomarkers of AA amyloidosis or of AL amyloidosis.

59. The method of claim 1, comprising assaying at least three biomarkers of AA amyloidosis or of AL amyloidosis.

60. The method of claim 1, wherein said biomarkers of multiple organ amyloidosis are selected from the group consisting of:
a variant serum amyloid A protein (SAA) allele;
SAA level;
C-reactive protein (CRP) level;
glycosaminoglycan (GAG) level;
interleukin-18 (IL-18) level;
macrophage-colony stimulating factor (M-CSF) level;
hepatocyte growth factor (HGF) level;
an antibody against citrulinated vimentin (Sa);
a monoclonal immunoglobulin light chain;
serum albumin level; and
creatinine clearance.

61. The method of claim 1, wherein said multiple organ amyloidosis is AA amyloidosis.

62. The method of claim 1, wherein said multiple organ amyloidosis is AL amyloidosis.

63. A method of assessing whether a subject has or is at risk of having AA amyloidosis comprising assessing at least two AA amyloidosis biomarkers selected from the group consisting of: a variant serum amyloid A protein (SAA) allele;
SAA level;
C-reactive protein (CRP) level;
glycosaminoglycan (GAG) level;
interleukin-18 (IL-18) level;
macrophage-colony stimulating factor (M-CSF) level;
hepatocyte growth factor (HGF) level; and
an antibody against citrulinated vimentin (Sa);
wherein abnormal levels of at least two of said AA amyloidosis biomarkers is indicative of the subject having or being at risk of having AA amyloidosis.

64. The method of claim 63, comprising detecting at least two of the following:
presence of a variant serum amyloid A protein (SAA) allele;
an elevated SAA level;
an elevated C-reactive protein (CRP) level;
a depressed glycosaminoglycan (GAG) level;
an elevated interleukin-18 (IL-18) level;
an elevated macrophage-colony stimulating factor (M-CSF) level;
an elevated hepatocyte growth factor (HGF) level; and
an antibody against citrulinated vimentin (Sa),
wherein detection of the presence or detection of said levels of least two of said AA amyloidosis biomarkers is indicative of the subject having or being at risk of having AA amyloidosis.

65. The method of claim 64, wherein a depressed GAG concentration in urine is less than about 5 mg/L.

66. The method of claim 63, comprising assaying at least three of said AA amyloidosis biomarkers.

67. The method of claim 66, comprising detecting:
an elevated SAA level,
a depressed glycosaminoglycan (GAG) level, and
an elevated hepatocyte growth factor (HGF) level.

68. The method of claim 67, further comprising detecting at least one additional biomarker selected from the group consisting of: an elevated interleukin-18 (IL-18) level; an elevated macrophage-colony stimulating factor (M-CSF) level; an elevated hepatocyte growth factor (HGF) level; and an antibody against citrulinated vimentin (Sa).

69. The method of claim 66, comprising detecting:
an elevated SAA level,
a depressed glycosaminoglycan (GAG) level, and
an antibody against citrulinated vimentin (Sa).

70. The method of claim 69, further comprising detecting an elevated hepatocyte growth factor (HGF) level.

71. The method of claim 66, comprising detecting:
a depressed glycosaminoglycan (GAG) level,
an elevated hepatocyte growth factor (HGF) level; and
an antibody against citrulinated vimentin (Sa).

72. The method of claim 71, further comprising, detecting an elevated level of SAA.

73. The method of claim 66, comprising detecting:
an elevated SAA level,
an elevated hepatocyte growth factor (HGF) level; and
an antibody against citrulinated vimentin (Sa).

74. The method of claim 73, further comprising detecting a depressed GAG level.

75. A method of monitoring treatment of a subject with multiple organ amyloidosis (MOA), comprising monitoring a level and/or a presence of at least two biomarkers selected from the group consisting of:
a variant serum amyloid A protein (SAA) allele;
SAA level;
C-reactive protein (CRP) level;
glycosaminoglycan (GAG) level;
interleukin-18 (IL-18) level;
macrophage-colony stimulating factor (M-CSF) level;
hepatocyte growth factor (HGF) level;
an antibody against citrulinated vimentin (Sa);
a monoclonal immunoglobulin light chain;
serum albumin level; and
creatinine clearance.
wherein a change in the level and/or a presence of said at least two biomarkers indicates a modulation of multiple organ amyloidosis (MOA) in the subject.

76. An assay kit for diagnosing multiple organ amyloidosis (MOA) or a risk of developing multiple organ amyloidosis (MOA), said kit comprising at least two of the following assays: an HGF ELISA assay, an ELISA capable of detecting SAA, an elevated C-reactive protein (CRP) assay, a heparin sulfate colorimetric kit, a urine GAG kit, an IL-18 ELISA assay, an M-CSF ELISA assay, an assay capable of detecting an antibody against Sa, an assay capable of detecting a variant SAA allele, an immunoglobulin light chain ELISA assay, a urine albumin kit, and a serum creatinine kit.
